SEQUENCE LISTING (6 sequences)

SEQ ID NO: 1 - DNA, 28 bp, synthetic construct
  source 1..28: mol_type = other DNA
atatgaatggggcaccgttgagctacag

SEQ ID NO: 2 - DNA, 18 bp, synthetic construct
  source 1..18: mol_type = other DNA
tcgagctctgcctggcgc

SEQ ID NO: 3 - DNA, 25 bp, synthetic construct
  source 1..25: mol_type = other DNA
aataagagtgcgggcgtggaagctc

SEQ ID NO: 4 - DNA, 20 bp, synthetic construct
  source 1..20: mol_type = other DNA
tccaaaatattgttcaccag

SEQ ID NO: 5 - DNA, 28 bp, synthetic construct
  source 1..28: mol_type = other DNA
gtttgtcaacgcctactcctcatgcttc

SEQ ID NO: 6 - DNA, 18 bp, synthetic construct
  source 1..18: mol_type = other DNA
tggaacaagaacatcttc

(19) United States
(12) Patent Application Publication
(10) Pub. No.: US 2024/0122922 A1
(43) Pub. Date: Apr. 18, 2024

(54) Methods Of Treating Cancer With Transmembrane Protein 16F (TMEM16F) Inhibitors

(71) Applicant: Institute For Cancer Research d/b/a The Research Institute of Fox Chase Cancer Center, Philadelphia, PA (US)

(72) Inventors: Igor Astsaturov, Philadelphia, PA (US); Edna Cukierman, Philadelphia, PA (US)

(21) Appl. No.: 18/473,540

(22) Filed: Sep. 25, 2023

Related U.S. Application Data
(60) Provisional application No. 63/410,067, filed on Sep. 26, 2022.

(51) Int. Cl. A61K 31/498; A61K 31/167; A61K 31/426; A61K 45/06; A61P 35/00

(52) U.S. Cl. CPC A61K 31/498; A61K 31/167; A61K 31/426; A61K 45/06; A61P 35/00

(57) ABSTRACT

Compositions comprising a TMEM16F inhibitor, an anti-cancer agent, and a pharmaceutically acceptable agent, and methods of treating of cancer comprising administering a TMEM16F inhibitor are provided herein.

### REFERENCE TO GOVERNMENT GRANTS

This invention was made with government support under CA231252 awarded by the National Institutes of Health. The government has certain rights in the invention.

### REFERENCE TO SEQUENCE LISTING

This application includes a Sequence Listing filed electronically as an XML file named 853003354SEQ, created on Sep. 25, 2023, with a size of 7000 bytes. The Sequence Listing is incorporated herein by reference.

### FIELD

The present disclosure is directed, in part, to compositions comprising a Transmembrane Protein 16F (TMEMT6F) inhibitor, an anti-cancer agent, and a pharmaceutically acceptable agent, and methods of treating cancer with a TMEM16F inhibitor.

### BACKGROUND

Cancer cells may derive their resistance to therapy and aggressive clinical course from the symbiotic signaling and metabolic interactions with cancer-associated fibroblasts (CAFs). CAFs have been shown to provide nutrients to cancer cells, specifically parasitic PDAC cells, including water soluble glucose and amino acids. In hypoxic tumor microenvironments, aggressive cancer cells are functional auxotrophs for lipids, and scavenge exogenous lipids to build cellular membranes. However, the mechanism by which cancer cells obtain these water-insoluble essential membrane building materials remains poorly understood.

TMEM16F (also known as anoctamin 6; ANO6) is a tissue-specific transmembrane Ca²⁺-dependent channel for phospholipids. TMEM16F plays a role in platelet activation, bone formation, T cell activation, viral infection, and phospholipid scrambling. Specifically, TMEM16F is further characterized as a Ca²⁺-activated phospholipid scramblase (CaPLSase) responsible for phosphatidylserine (PS) externalization from the inner to the outer leaflet of the plasma membrane.

### SUMMARY

The present disclosure provides compositions comprising a TMEM16F inhibitor, an anti-cancer agent, and a pharmaceutically acceptable carrier.

The present also disclosure provides methods treating cancer in a mammal, the methods comprising administering a TMEM16F to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows delivery of low-density lipoprotein particles to pancreatic cancer cells is mediated by cancer-associated fibroblasts. Panel A shows intravital microscopy images of orthotopic pancreatic tumors at 15 minutes, 90 minutes, and 17 hours after intravenous injection of donor LDL labeled with BODIPY-conjugated cholesterol. Panel B shows total integrated intensity of BODIPY-cholesterol over time. Data are represented as mean±SEM. ***, p<0.001 as compared with 15 minutes post injection (unpaired two-tailed t-test). Panel C shows co-localization of BODIPY-cholesterol with DsRed-expressing carcinoma cells over time. Graph represents pooled data from 2-3 anesthetized animals imaged at 1-2-hour intervals by multiphoton microscopy. Panel D shows flow cytometry enumeration of LDL label in subsets of cells from disintegrated tumors 90 minutes post intravenous administration of fluorescent LDL. Data are represented as mean±SEM. **, p<0.01, ***, p<0.001 as compared with CAFs (unpaired two-tailed t-test). Panel E shows enhanced intravital images of endosomal aggregates containing BODIPY-cholesterol and cancer cells in pancreatic tumors. Red, KPCN349 murine pancreatic carcinoma cells expressing DsRed; green, BODIPY-conjugated cholesterol; blue, second harmonic generation collagen.

FIG. 2 shows gating strategy to enumerate LDL uptake in orthotopic pancreatic tumors. Approximately 90 minutes post intravenous injection via retroorbital vein of 150 μL of fluorescent LDL particles, animals were euthanized and single-cell suspension obtained using Miltenyi Biotec GentleMacs dissociator in gentleMACS C Tubes (#130-093-23) and mouse tumor tissue dissociation kit (#130-096-730) as per the manufacturer's instructions followed by washes and filtering of non-cellular debris. The cells were first gated using FSC-A and SSC-A plot to eliminate small non-cellular particles and aggregates. LDL-positive population (˜10%) was selected based on a cut off for vehicle-injected negative controls. GFP- or DsRed-positive carcinoma cells were separated from CD45+ and CD11b+(myeloid/immune) and PDGFRa+(CAFs) populations.

FIG. 3 shows transfer of membrane cholesterol from cancer-associated fibroblasts to cancer cells via trogocytosis. Panel A shows flow cytometry measurement of uptake of CAFs membranes by KPCN349 cells in co-culture. CAFs were labeled with BODIPY-cholesterol and KPCN349 cells with CPD670 and plated at 1:1 ratio for the indicated times. Panel B shows uptake of BODIPY-cholesterol by KPCN349 cells in co-culture occurs via cell contacts with CAFs. Data are represented as mean±SEM. *, p<0.05 as compared with KCN349 cells making no contacts (unpaired two-tailed t-test). Panel C shows representative image of CAF membrane uptake by KPCN349 PDAC cells (red, CPD670) and CAFs (green, BODIPY-cholesterol). Panel D shows fold change in cell numbers of KPC (Nsdhl-proficient) or KPCN (Nsdhl-deficient) mouse pancreatic adenocarcinoma cells in the absence of exogenous lipids in 5% lipid-depleted serum (LDS). Data are represented as mean±SEM. *, p<0.05, **, p<0.01, ****, p<0.0001 as compared with cells grown in 5% FBS (unpaired two-tailed t-test). Panel E shows apoptosis in LDS estimated by Annexin-V/FITC labeling of Nsdhl-deficient KPCN349 cells is rescued by co-culture with CAFs, or by addition of 50 μM LDL cholesterol. CAF/TW, co-culture of CAFs and cancer cells in transwell plates; CM, CAF-conditioned 5% LDS/DMEM media for 72 hours. Data are represented as mean±SEM. *, p<0.05,**, p<0.01, ****, p<0.0001 as compared with KPCN349 cells in LDS (Wilcoxon-test). Panel F shows inverse relationship of Annexin-V-positive carcinoma cells and BODIPY-cholesterol acquired from labeled CAFs in co-cultures. CAFs and KPCN349 cells were plated at varying densities 72 hours prior to Annexin-V labeling. Panel G shows 10-day colony formation by mouse pancreatic carcinoma cells in the indicated conditions. Panel H shows quantification of the colony areas as in Panel C. Data are represented as mean±SEM. *, p<0.05,**, p<0.01, ****, p<0.0001 as compared with KPCN349 cells in LDS (Mann Whitney-test). Panel I shows flow cytometry measurement of uptake of human HLA by KPCN349 cells in co-culture. Human CAFs were labeled with BODIPY-cholesterol and KPCN349 cells with CPD670 and plated at 1:1 ratio for 8 hours. Panel J shows masking of cell surface phosphatidylserine with recombinant annexin V or bis-Zn-dipicolylamine antagonizes uptake of CAF membranes by KPCN349 cells. CAFs and KPCN349 cells were labeled as in Panel F. Annexin V and Zn-DPA were added to co-culture media. Data are represented as mean±SD. *, p<0.05 as compared with vehicle (unpaired two-tailed t-test).

FIG. 4 shows transfer of membrane cholesterol from cancer-associated fibroblasts to cancer cells via trogocytosis. Panel A shows quantification of PDAC-CAF cell contacts duration in co-cultures. Data are compiled from two independent time-lapse microscopy experiments. Contacts durations are inferred from the number of sequential 15-minute frames during which the contacts were maintained. The contact duration data were grouped at 2-hour intervals for simplicity. Panel B shows 3D reconstruction of a Z-stack images of CAF (green) and KPCN349 (pink) interface taken at 2-second intervals. Arrow, emerging CAF membrane protrusion towards the body of carcinoma cells (trogocytosis). Panel C shows quantification of cholesterol concentration (μM) in 10% fetal bovine serum (FBS) in DMEM and in 10% LDS/DMEM media conditioned with CAFs for 3 days. Data are represented as mean±SEM of independent experiments. Panel D shows viability of cholesterol auxotroph cell line KPCN349 (NSDHL-null) in 10% LDS supplemented with varying concentrations of cholesterol added to 72 hours culture in the form of human donor LDL. Panel E shows the effect of cell plating density on the rate of BODIPY-cholesterol uptake by KPCN349 cells in 24 hours. Panel F shows the effects of temperature (left panel), or extracellular calcium and magnesium (right panel) on CAF membrane trogocytosis by Panc-1 cells co-cultured with CAFs in 10% FBS DMEM (middle panel), or in CAF-conditioned media (CM, middle panel). Adherent CAFs were stained 1 μM PKH67 (Sigma-Aldrich), Panc-1 were stained in suspension with 10 μM CellTracker™ orange CMTMR (Thermo Fisher Scientific) and added to co-cultures with CAFs for 3 hours. The uptake of PKH67 by Panc-1 cells was analyzed by flow cytometry.

FIG. 5 shows high expression of ANO6 in pancreatic adenocarcinoma confers poor survival. Panel A shows ANO gene family transcripts expression visualization by single-cell RNA sequencing of 17,205 single cells isolated from four advanced mouse KPC pancreatic tumors. Panel B shows ANO gene family transcripts expression visualization by single-cell RNA sequencing of 20,929 single cells isolated from five human pancreatic tumors. In Panel A and Panel B, the circle size represents percentage of indicated cells and color intensity represents Z-score normalized expression of the genes. Panel C shows comparison of overall survival of stage 1-3 PDAC patients from the top and the bottom quartiles of ANO6 protein expression obtained from the NCI Clinical Proteomic Tumor Analysis Consortium (CPTAC) dataset. Panel D shows comparison of overall survival of stage 1-3 PDAC patients by ANO6 mRNA expression above or below the fragments per kilobase of exon per million mapped fragments (FPKM) cut off 15.36. Data was obtained from the NCI TCGA program. In Panel C and Panel D, p-values by Mantel-Cox log-rank test. Panel E shows ANO6 expression by western blot in protein lysates from fibroblastic cell lines obtained from PDAC tumor (T) or adjacent non-malignant pancreatic tissues (N).

FIG. 6 shows transcriptome analyses of ANO6 expression in mouse PDAC models and human cancers. Panel A shows cell type determination based on uniform manifold approximation and projection (UMAP) embedding of transcriptomes of 17,205 single cells isolated from 4 KPPC advanced tumors using the 10× Genomics single-cell RNA sequencing platform. Nine cell types were identified by graph-based clustering are indicated by color. Panel B shows heat map of differentially expressed genes for each cluster. Z-score normalized expression of the enriched genes for each cluster is shown as a log 2-fold change in cells within a cluster relative to all other cells in the dataset. Panel C shows UMAP-embedding of transcriptomes of 20,929 single cells from 5 human pancreatic adenocarcinoma tumors. Raw sequencing data for CRR034503-CRR034507 were downloaded and processed as described (Gabitova et al., Clin. Cancer Res., 2014, 20, 28-34). Nine cell types were identified by graph-based clustering are indicated by color. Panel D shows heat map of differentially expressed genes for each cluster. Z-score normalized expression of the enriched genes for each cluster are shown as a log 2-fold change in cells within a cluster relative to all other cells in the dataset. Abbreviations: iCAF, inflammatory cancer associated fibroblasts; myCAF, myofibroblasts; Msln+CAF, mesothelin-positive CAFs. Panel E shows the effect of ANO6 transcript expression on overall survival of all human cancers (left, TCGA Pan-Cancer bulk RNA sequencing dataset), or selectively in breast invasive carcinoma (middle, TCGA BRCA bulk RNA sequencing dataset), and cervical carcinoma (right, TCGA CESC bulk RNA sequencing dataset).

FIG. 7 shows ANO6 regulates cholesterol metabolism and tumor-promoting function of CAFs. Panel A shows growth of ANO6-KD fibroblasts in vitro by continuous xCELLigence impedance assay. Insert, western blot demonstrating loss of ANO6 in CRISPRi-modified C7 CAFs. Panel B shows representative images of CRISPRi-control and ANO6-KD C7 fibroblasts in phase contrast (top) or Nile red (bottom) labeling of lipid droplets. Panel C shows quantification of Nile red fluorescence in lipid droplets. Data are represented as mean±SD fluorescence intensity relative to controls obtained from 4 or more technical replicates in 3 independent experiments. ***, p<0.001; ****, p<0.0001 (unpaired two-sided t-test). Panel D shows expression of indicated protein epitopes in total cellular lysates of C7 CRISPRi-control and C7 ANO6-KD fibroblasts. Bands density relative to control C7 lysates is shown under each lane. Panel E shows viability of cholesterol auxotroph MiaPaCa^{ΔNSDHL} cells (CRISPRi-depleted of NSDHL) cultured for 4 days in 10% LDS alone, or in the presence of C7 CAFs, or C7 ANO6-KD. Data represent fluorescence of DsRed tagged MiaPaCa^{ΔNSDHL} cells relative to co-cultures with control CAFs. Each element represents individual technical replicate. ****, p<0.0001 (unpaired two-sided t-test). Panel F shows viability of cholesterol auxotroph Panc-1^{ΔNSDHL} cells (CRISPRi-depleted of NSDHL) cultured for 4 days in 10% LDS alone or in the presence of C7 CAFs, ANO6-KD, or ANO6-KD modified with lentiviral expression of a full-length murine ANO6-mCherry, or with indicated mutations. Data represent fluorescence of Panc-1^{ΔNSDHL} cells pre-labeled with Hoechst-33342 relative to co-cultures with control CAFs. Each element represents individual technical replicate. *, p<0.05, **, p<0.001 (unpaired two-sided t-test). Panel G shows tumor weights at 5 weeks following orthotopic implantations of 5*105 Panc-1^{ΔNSDHL} cells alone or with ANO6-modified CAFs at 1:3 ratio. Panel H shows normalized enrichment scores by for select (FDR<0.25) Hallmark signatures in ANO6-KD fibroblasts relative to the CRIPSRi-controls. Panel I shows hallmark cholesterol homeostasis signature enrichment plots for ANO6-KD and controls of C7 and 37 lines of CAFs. Panel J shows LC-MS profiling of cholesterol esters in ANO6-KD and CRISPRi-controls. Data are represented as mean±SD of 3 independent replicates of measurement. *, p<0.05, **, p<0.01 (unpaired two-sided t-test). Panel K shows a Volcano plot of lipids enriched in Panc-1 cells co-cultured for 24 hours with CRISPRi-control or ANO6-KD C7 CAFs. Phosphatidylserine (40:7) enriched in Panc-1 cells co-cultured with control CAFs relative to ANO6-KD is marked in blue. Significant changes are defined as FC>2 and p<0.05 (unpaired two-sided t-test).

FIG. 8 shows ANO6 regulates cholesterol metabolism and tumor-promoting function of CAFs. Panel A shows cell area of ANO6-KD fibroblasts relative to CAF-Control C7 line. Data represents mean±SEM of cell area measured using Image J of phase contrast images from 5 in dependent cultures; **, p<0.01 (unpaired two-tailed t-test). Panel B shows alignment of extracellular matrix fibers produced in vitro by C7-Control and C7 ANO6-KD fibroblasts. Anti-fibronectin was used to label fibers of extracellular matrix; the fiber angle distribution was determined by ‘Orientation J’ plugin in Image J. Images represent normalized values of fibers angles as shown in the colored bar on the right. Plotted data (Panel C) represents percentages of fibronectin fibers aligned at less or equal 15° angles for both conditions as mean±SD. ***, p<0.001 as compared to control CAFs (unpaired two-tailed t-test). Panel D shows viability of cholesterol auxotroph Panc-1^{ΔNSDHL} DsRed-tagged cancer cells in lipid-depleted serum (5% LDS) co-cultured with CRISPRi-Control or ANO6-KD C7 fibroblasts. Data are presented as mean±SEM of ratio of dead (SYTOX Blue-positive) and live (DsRed-positive) Panc-1 cells. Fluorescent images were acquired on automated high throughput ImageXpress (Molecular Devices) microscope and analyzed using MetaXpress software. Panel E shows PtdSer externalization induced by addition of ionomycin (10 μM, 5 minutes) or under basal culture conditions (Panel F) assessed by intravital annexin XII staining of CAF membranes of C7 control CAFs, C7 ANO6-KD, or ANO6-KD plus wild-type murine ANO6-mCherry (calcium-responsive), or with indicated modifications: D409G (constitutively active), D703R (inactive due to calcium-domain mutation), and Y563A (constitutively active). Graph represents total annexin XII fluorescence intensity represented as mean±SD. ***, p<0.001, ****, p<0.0001 as compared with control CAFs treated with ionomycin (unpaired two-tailed t-test). Panel G shows CRIPSRi-mediated silencing of NADP-sterol-dehydrogenase-like (NSDHL) in Panc-1 PDAC cells results in accelerated tumor growth. Representative images of pancreatic tumors at 5 weeks post implantation of 10⁵ of cancer cells to pancreatic tails of C.B-17.icr SCID mice. Panel H shows weights of CRISPRi-control and NSDHL-KD Panc-1 tumors. Data are presented as mean±SD from n=6 tumors per condition; ***, p<0.001 (unpaired two-tailed t test). Panel I shows quantification of Nile red fluorescence of lipid droplets in C7 control CAFs, C7 ANO6-KD, or ANO6-KD plus wild-type murine ANO6-mCherry, or with indicated modifications D409G, D703R, and Y563A. Data are represented as mean±SD of 5 technical replicates combined from 2 independent experiments. **, p<0.01; ***, p<0.001; **** p<0.0001 (unpaired two-sided t-test).

FIG. 9 shows blockade of ANO6 restrains growth and blocks delivery of exogenous lipids to pancreatic tumors. Panel A shows treatment schema of mice carrying KPC3 syngeneic pancreatic tumor grafts. Animals were given 3 weekly cycles of niclosamide 150 mg/kg/day, clofazimine 50 mg/kg/day, or vehicle followed by intravital imaging and tumor collection. Weights of orthotopic syngeneic pancreatic tumors following treatment with oral ANO6 inhibitors niclosamide (Panel B) or clofazimine (Panel C) at 3 weeks post implantation are shown. Data are represented as mean±SD. ***, p<0.001 comparing with vehicle-treated mice (unpaired two-tailed t-test). Panel D shows intravital imaging quantification of intratumoral and peri-tumoral BODIPY-cholesterol fluorescence in DsRed-tagged KPC3 tumors at 10, 16 and 19 hours following intravenous administration of labeled LDL. Data are represented as mean±SD of ratios of intratumoral to peri-tumoral BODIPY-cholesterol fluorescence. *, p<0.05 Panel E shows rRepresentative intravital images of vesicles containing BODIPY-cholesterol within orthotopic DsRed-tagged KPC3 tumors treated with niclosamide or vehicle. Panel F shows quantification of intratumoral vesicles as in Panel E. Cumulative data from 2 independent imaging experiments are represented as mean±SD of vesicle volumes enumerated from multiple tumor areas in 2-3 mice at each time point, each element represents vesicles volume in a tumor area. *, p<0.05 (unpaired two-tailed t-test).

FIG. 10 shows lockade of ANO6 restrains growth and blocks delivery of exogenous cholesterol to pancreatic tumors. Shown are images of individual pancreatic tumors collected at 3 weeks post implantation of 10⁵ of KPC3 PDAC cells to pancreatic tails. Animals were treated with niclosamide (Panel A) or clofazimine (Panel B) as indicated in FIG. 9, Panel A.

FIG. 11 shows CAFs KD TMEM16F without membrane ruffling compared to CAFs KD Ctrl.

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” of “one or more” unless the context clearly indicates otherwise.

As used herein, the term “about” means that the numerical value is approximate and small variations would not significantly affect the practice of the disclosed embodiments. Where a numerical value can vary ±10% and remain with the scope of the disclosed embodiments.

As used herein, the terms “comprising” (and any form of comprising, such as “comprise”, “comprises” and “comprised”), “having” (and any form of having, such as “have” and “has”), “including” (and any form of including, such as “contains” and “contain”), are inclusive or open-ended and do not exclude additional, unrecited elements of method steps.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. For example, a subject who receives treatment with a TMEM16F inhibitor, as described herein, to treat cancer is “in need thereof” (i.e., as opposed to receiving a TMEM16F inhibitor to treat parasitic worms).

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal of a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the terms “treat,” “treated,” or “treating” mean therapeutic treatment wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

Recently, it has been discovered (as described herein) that pancreatic ductal adenocarcinoma (PDAC) cells utilize cancer-associated fibroblasts (CAFs) as a main source of lipids in vivo through trogocytosis. In turn, CAFs upregulate phosphatidylserine (PtdSer) on the outer leaflet of the plasma membrane (PM). Furthermore, Ca²⁺-dependent phospholipid scramblase TMEM16F is a critical regulator of PtdSer externalization and is highly expressed in human PDAC CAFs compared to fibroblasts isolated from matching adjacent non-malignant pancreatic tissues CAFs deficient in TMEM16F scramblase. These observations led to the examination, as described herein, of the possible functions of trogocytosis as a new mode of lipid scavenging by PDAC cells from CAFs involving activation of Ca²⁺-dependent phospholipid scramblase TMEM16F in CAFs and increased expression of PtdSer as “eat me” signals on CAF PM. In the studies described herein, PtdSer externalization was significantly inhibited by treatment with TMEM16F inhibitor, indicating that TMEM16F inhibitors could be used to treat cancer.

The present disclosure provides pharmaceutical compositions comprising a TMEM16F inhibitor, an anti-cancer agent, and a pharmaceutically acceptable carrier.

In any of the embodiments described herein, the TMEM16F inhibitor may be niclosamide, clofazimine, nitazoxanide, hexachlorophene, 10 bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof. In any of the embodiments described herein, the TMEM16F inhibitor is niclosamide, clofazimine, or nitazoxanide, or any combination thereof. In some embodiments, the TMEM16F inhibitor is niclosamide. In some embodiments, the TMEM16F inhibitor is clofazimine. In some embodiments, the TMEM16F inhibitor is nitazoxanide. In some embodiments, the TMEM16F inhibitor is hexachlorophene. In some embodiments, the TMEM16F inhibitor is 10 bm. In some embodiments, the TMEM16F inhibitor is Monna. In some embodiments, the TMEM16F inhibitor is Ani9. In some embodiments, the TMEM16F inhibitor is Ani9 derivative 5f. In some embodiments, the TMEM16F inhibitor is tannic acid. In some embodiments, the TMEM16F inhibitor is T16A-A01. In some embodiments, the TMEM16F inhibitor is dichlorophen. In some embodiments, the TMEM16F inhibitor is idebenone. In some embodiments, the TMEM16F inhibitor is shikonin. In some embodiments, the TMEM16F inhibitor is benzbromarone. In some embodiments, the TMEM16F inhibitor is CaCC-A01. In some embodiments, the TMEM16F inhibitor is 9-phenanthrol. In some embodiments, the TMEM16F inhibitor is niflumic acid. In some embodiments, the TMEM16F inhibitor is flufenamic acid. In some embodiments, the TMEM16F inhibitor is talniflumate. In some embodiments, the TMEM16F inhibitor is A9C. In some embodiments, the TMEM16F inhibitor is dehydroandrographolide. In some embodiments, the TMEM16F inhibitor is DIDS. In some embodiments, the TMEM16F inhibitor is NPPB.

In any of the embodiments described herein, the TMEM16F inhibitor may be an analog of niclosamide (see, Shamim et al., Bioorg. Med. Chem. Lett., 2021, 40, 127906).

In any of the embodiments described herein, the TMEM16F inhibitor may be present in amount from about 1 mg to about 1000 mg, from about 50 mg to about 800 mg, from about 75 mg to about 600 mg, from about 100 mg to about 500 mg, or form about 200 to about 400 mg.

In any of the embodiments described herein, the anti-cancer agent may be methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, or docetaxel, or any combination thereof. In some embodiments, the anti-cancer agent is methotrexate. In some embodiments, the anti-cancer agent is taxol. In some embodiments, the anti-cancer agent is mercaptopurine. In some embodiments, the anti-cancer agent is thioguanine. In some embodiments, the anti-cancer agent is hydroxyurea. In some embodiments, the anti-cancer agent is cytarabine. In some embodiments, the anti-cancer agent is cyclophosphamide. In some embodiments, the anti-cancer agent is ifosfamide. In some embodiments, the anti-cancer agent is a nitrosourea. In some embodiments, the anti-cancer agent is cisplatin. In some embodiments, the anti-cancer agent is carboplatin. In some embodiments, the anti-cancer agent is mitomycin. In some embodiments, the anti-cancer agent is dacarbazine. In some embodiments, the anti-cancer agent is procarbazine. In some embodiments, the anti-cancer agent is an etoposide. In some embodiments, the anti-cancer agent is a campathecin. In some embodiments, the anti-cancer agent is bleomycin. In some embodiments, the anti-cancer agent is doxorubicin. In some embodiments, the anti-cancer agent is idarubicin. In some embodiments, the anti-cancer agent is daunorubicin. In some embodiments, the anti-cancer agent is dactinomycin. In some embodiments, the anti-cancer agent is plicamycin. In some embodiments, the anti-cancer agent is mitoxantrone. In some embodiments, the anti-cancer agent is an asparaginase. In some embodiments, the anti-cancer agent is vinblastine. In some embodiments, the anti-cancer agent is vincristine. In some embodiments, the anti-cancer agent is vinorelbine. In some embodiments, the anti-cancer agent is paclitaxel. In some embodiments, the anti-cancer agent is docetaxel.

In any of the embodiments described herein, the pharmaceutical composition is an oral dosage formulation, an intravenous dosage formulation, a topical dosage formulation, an intraperitoneal dosage formulation, or an intrathecal dosage formulation.

In some embodiments, the pharmaceutical composition is an oral dosage formulation in the form of a pill, tablet, capsule, cachet, gel-cap, pellet, powder, granule, or liquid.

In some embodiments, the pharmaceutical composition is protected from light and present within a blister pack, bottle, or intravenous bag.

The compounds and compositions described herein can be formulated for parenteral administration by injection, such as by bolus injection or continuous infusion. The compositions can take such forms as suspensions, solutions or emulsions in oily or aqueous vehicles, and can contain formulary agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

The compounds and compositions described herein can also be formulated as a depot preparation. Such long-acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The present disclosure also provides methods of treating cancer in a mammal, the methods comprising administering a TMEM16F inhibitor to the mammal, or a composition comprising a TMEM16F inhibitor and an anti-cancer agent, as described herein. In such methods, any of the TMEM16F inhibitors, or any combination thereof, or compositions comprising a TMEM16F inhibitor and an anti-cancer agent, as described herein, can be used.

In some embodiments, the cancer is lung cancer, breast cancer, prostate cancer, ovarian cancer, testicular cancer, colon cancer, renal cancer, bladder cancer, pancreatic cancer, glioblastoma, neuroblastoma, retinoblastoma, leukemia, melanoma, kidney or renal cancer, or osteosarcoma. In some embodiments, the cancer is lung cancer. In some embodiments, the cancer is breast cancer. In some embodiments, the cancer is prostate cancer. In some embodiments, the cancer is ovarian cancer. In some embodiments, the cancer is testicular cancer. In some embodiments, the cancer is colon cancer. In some embodiments, the cancer is renal cancer. In some embodiments, the cancer is bladder cancer. In some embodiments, the cancer is pancreatic cancer. In some embodiments, the cancer is glioblastoma. In some embodiments, the cancer is neuroblastoma. In some embodiments, the cancer is retinoblastoma. In some embodiments, the cancer is leukemia. In some embodiments, the cancer is melanoma. In some embodiments, the cancer is kidney or renal cancer. In some embodiments, the cancer is osteosarcoma.

In some embodiments, the mammal is also administered radiation therapy.

In some embodiments, the mammal is human.

The compounds and compositions described herein can be administered by any route of administration including, but not limited to, oral, sublingual, buccal, rectal, intranasal, inhalation, eye drops, ear drops, epidural, intracerebral, intracerebroventricular, intrathecal, epicutaneous or transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous or transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The compounds and compositions can be administered by continuous infusion subcutaneously over a period of about 15 minutes to about 24 hours.

For administration by inhalation, the compounds and compositions described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In some embodiments, the compounds and compositions can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds and compositions described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The amount of compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

In some embodiments, it may be desirable to administer one or more compounds, or a pharmaceutically acceptable salt thereof, or compositions comprising the same, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as silastic membranes, or fibers). Formulations for injection can be presented in unit dosage form, such as in ampoules or in multi-dose containers, with an added preservative.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: General Methodology

### Animal Studies

For the xenograft study, anesthetized 6-8-week-old C.B-17.icr SCID mice (Taconic, NY) were injected to the tail of pancreas with 5×10⁵ of NSDHL-depleted Panc-1 cells (CRISPRi-mediated knockdown) in 30 μL of serum-free DMEM. For co-implantation with CAFs, 5×10⁵ Panc-1 cells were mixed at 1:3 ratio with 1.5×10⁶ of C7 CAFs in 30 μL of serum-free DMEM.

Syngeneic 5×10⁵ KPC3 cells were orthotopically injected to the tail of pancreas of anesthetized C57Bl/6J mice. Treatment with ANO6 inhibitors niclosamide or clofazimine commenced on 2 days post-implantation: niclosamide (150 mg/kg of mouse body weight, Cayman #10649), clofazimine (50 mg/kg, MedChem Express), or corn oil (50 mg/kg, Sigma #C8267) as vehicle were given by oral gavages 5 days/week for 3 weeks. Mice were kept under defined-flora pathogen-free conditions. Mice of both genders, equally distributed, were used for the experiments.

### In Vivo Fluorescent Low Density Lipoprotein Imaging by Multiphoton Microscopy

Mice with established orthotopic tumors roughly 21 days post implantation were anesthetized with 1.5-2% isoflurane/oxygen mixture maintained for the duration of imaging. Pancreatic tumors were exposed through a 1 cm incision on the left flank of the mouse placed on a 350 heating pad (Kent Scientific). Areas of DsRed signal in tumor cells were selected for 3D confocal images using Leica SP8 Dive multiphoton imaging system (Leica Microsystems) with a 25× objective (Leica Microsystems) prior (to establish background fluorescence) to intravenous administration of 150 μL of LDL carrying BODIPY-cholesterol and at 0.5, 1, 5, 16, and 19-hour interval post injection with XY resolution 0.867 μm/px and Z distance 2 μm. For each time interval, 2-3 mice were imaged for 0.5-2 hours under continuous anesthesia. At the end of each imaging session, the animals were euthanized, and the tumor sample was collected. The acquired 3D stacks were analyzed using Imaris 10.0.0 software (Oxford Instruments, UK).

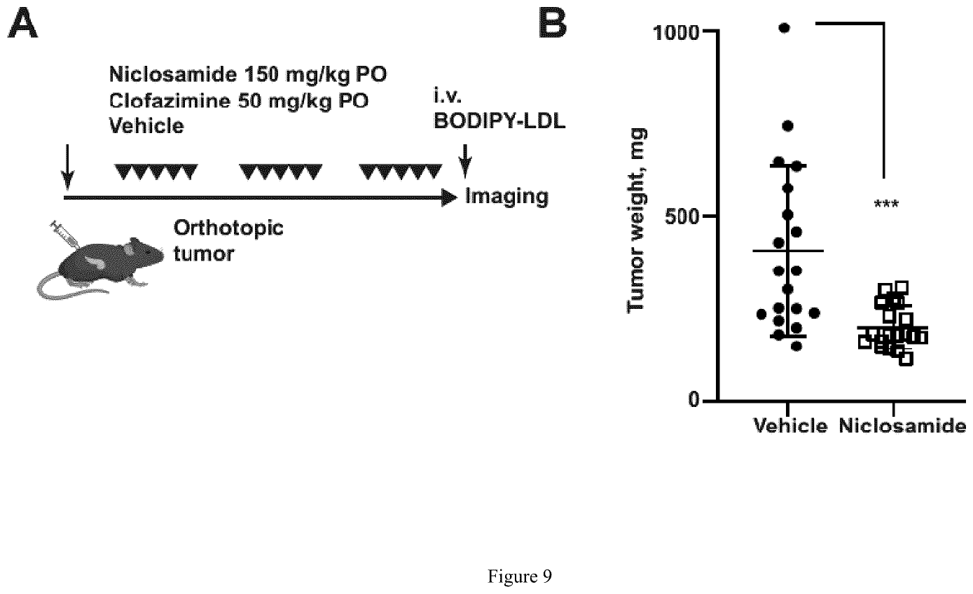

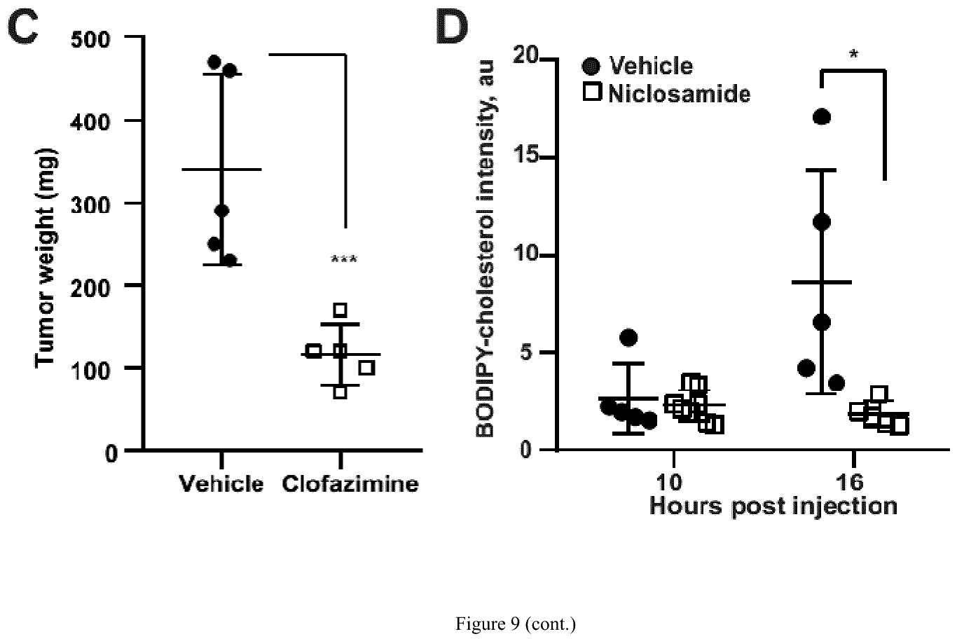

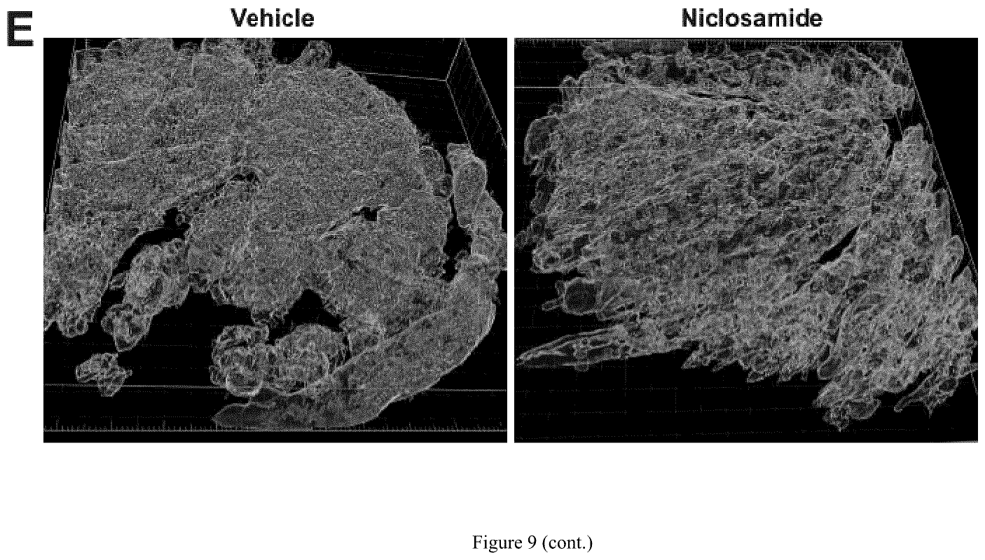

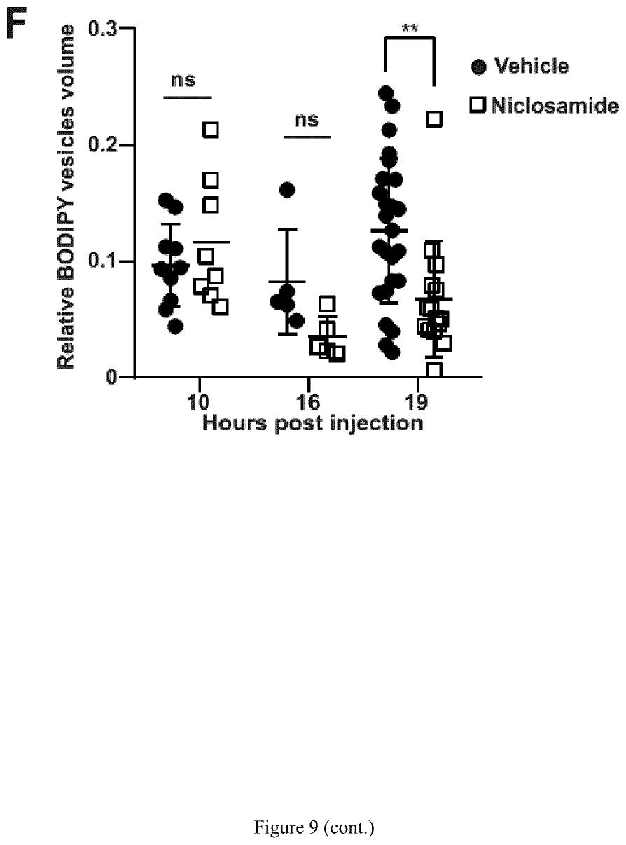

For measurements of BODIPY-cholesterol uptake by tumors in FIG. 9, Panel D, tumor volumes were defined using “surface option” in Imaris package with surface grain size 0.867 μm, intensity threshold and size exclusion 300 voxel to exclude non-specific speckles. Volume outside of the tumor within 20 μm of its boundary was defined using “distance transform” option in Imaris software. Cholesterol volume was identified using “surface option” in Imaris with surface grain size 0.867 um, automatic intensity threshold and size exclusion set at 10 voxel. Mean intensity of cholesterol volume colocalized with tumor volume or volume outside of tumor was measured and ratio inside intensity/outside intensity was calculated.

For measurements of BODIPY-cholesterol vesicles in tumors in FIG. 9, Panel E, tumor volumes were defined as above. The volumes of BODIPY-cholesterol-positive vesicles were defined using surface grain size 0.1 μm and background subtraction with 3 μm sphere, automatic intensity threshold and excluding objects below 26 voxels. The volume of vesicles inside the tumor were expressed as a percentage of tumor volume.

### Cell Lines

PDAC cells were obtained from ATCC and maintained in DMEM supplemented with 10% v/v FBS, 2 mM L-glutamine and 100 mg/ml Penicillin/Streptomycin. Murine pancreatic carcinoma cell lines were isolated as reported (Gabitova-Comell et al., Cancer Cell, 2020, 38, 567-583). All cell lines were regularly tested for Mycoplasma, as determined by PCR detection methods, and all lines tested negative. Human CAFs were isolated as described (Franco-Barraza J., Elife, 2017, 6) from the residuals of PDAC surgical samples.

### Plasmid Transfections and CRISPRi-Mediated Gene Silencing

Transfection of GFP-Mapper (117721, Addgene, Watertown, MA), mCherry-ANO6 (62554, Addgene, Watertown, MA) or STIM1-YFP (19754, Addgene, Watertown, MA) were carried out by mixing 2.5 μg of plasmid DNA with 7.5 μl of TransIT-X2 reagent (Mirus, Madison, WI). The mixture was added to 10⁵ CAF or PDAC cells cultured in serum-free and antibiotic-free media overnight.

For CRISPRi, an all-in-one lentiviral CRISPRi plasmid containing a nuclease-dead Cas9 (dCas9) fused to the transcriptional repressor domain KRAB and a gene specific gRNA was used (Gilbert et al., Cell, 2013, 154, 442-451). The top 3 best predicted gRNA were chosen from the published study (Horlbeck et al., Elife, 2016, 5) and individually cloned into the all-in-one lentiviral vector, CRISPRi-Puro modified from the addgene plasmid #71236 (a gift from Charles Gersbach (Thakore et al., Nat. Methods, 2015, 12, 1143-1149) containing a “stuffer” at the gRNA cloning site). Lentiviruses were generated by transfecting the CRISPRi-Puro plasmid, along with the packaging plasmids psPAX2 (a gift from Didier Trono; Addgene plasmid #12260) and pMD2.G (VSV-G envelope, a gift from Didier Trono; Addgene plasmid #12259) with X-tremeGene9 transfection reagent (#6365787001, Sigma-Aldrich) into 293T cells in serum free/antibiotic free media overnight. The following day, media was replaced with complete media containing FBS and media containing lentiviruses were collected at days 2 and 4 post-transfection. The lentiviral media was then filtered through a 0.45 μm filter (#HAWP14250, Millipore) and used to transduce target cells, by culturing cells in lentiviral media plus 10 μg/mL Polybrene (#sc-134220, Santa Cruz Biotechnology). After 24 hours, lentiviral media was replaced with complete media and cells were selected 48 hours later with puromycin (specific for each cell lines) for 14 days. Surviving cells were subsequently expanded and knockdown of target genes was confirmed at the protein and transcriptomic levels.

### Generation of ANO6-mCherry Mutant CAFs

Mutations in the mouse ANO6 sequence of plasmid ANO6-Plvx-mCherry-cl (Addgene #62554) were created using the Q5® Site-Directed Mutagenesis Kit (NEB E0554S). Primers were designed with the NEBaseChanger tool (see, world wide web at “nebasechangerv1.neb.com/”) and gradient gels were run to determine ideal PCR conditions. After kinase, ligase and dpnI (KLD) treatment of the PCR products, they were used to transform competent bacteria (NEB 5-alpha competent E. coli). Following plasmid extraction and purification, the intended substitution for each mutation was confirmed by Sanger sequencing (Azenta) and the mutated plasmids were transfected into HEK293T cells. The resulting viruses were filtered and transduced into CAF ANO6 CRISPRi-depleted cells. After growth and expansion, cell lines were selected by flow cytometry for mCherry expression.

### Mutation Primers

D409G forward-ATATGAATGGGGCACCGTTGAGCTACAG (SEQ ID NO:1)
   reverse-TCGAGCTCTGCCTGGCGC (SEQ ID NO:2)
D703R forward-AATAAGAGTGCGGGCGTGGAAGCTC (SEQ ID NO:3)
   reverse-TCCAAAATATTGTTCACCAG (SEQ ID NO:4)
Y563A forward-GTTTGTCAACGCCTACTCCTCATGCTTC (SEQ ID NO:5)
   reverse-TGGAACAAGAACATCTTC (SEQ ID NO:6)

### Analyses of Protein Expression by Western Blotting

Cultured cells were lysed in RIPA buffer (#24928, Santa Cruz) with phosphatase inhibitor (#1862495, Thermo Fisher Scientific) and protease inhibitors (#1861278, Thermo Fisher Scientific) on ice and cleared then by centrifugation (15 minutes at 17,000 g). The protein concentration was measured with a Pierce BCA Protein Assay Kit (#23225, Thermo Fisher Scientific). Proteins were separated on 4-12% Bis-Tris Protein gels (Invitrogen) and then horizontally transferred to the Immobilon-FL PVDF membrane (#IPFL00010, Millipore). Primary and secondary antibodies were used at the concentrations indicated below according to manufacturer's instructions. The density of obtained bands was quantified with Image J software (FIJI).

### Lipid Droplets Fluorescence Measurements

Lipids droplet staining was performed in PDAC or CAFs cells (5000 cells per well in a 96-well plate) with Lipid Droplets Fluorescence Assay Kit (Cayman #No. 500001 or AAT Bioquest #22730) according to the manufacturer's instructions. The fluorescence intensity was measured on a plate reader using the manufacturer's recommended settings.

### Cholesterol Measurement

Cholesterol level in PDAC or CAFs cells were measured by Amplex Red Kit (#A12216, Life Technologies). Briefly, 500,000 cells were seeded in a 6-well plate and cholesterol was measured in cell lysate or supernatant according to the manufacturer's instructions. BCA test was performed in parallel for normalization.

### Phosphatidylserine Externalization Assay

CAFs were seeded in 24-well glass bottom plates (#81156, Ibidi, Fitchburg, WI) at a density of 20,000 cells per well. Cells were then treated with niclosamide (1 μM) for 2 hours. After one wash with FBS-free media at RT, cells were incubated with 250 μl of 1:100 annexin XII and 1:200 propidium iodide (pSIVA Abcam #ab129817), with or without 10 μM ionomycin. Cells were then incubated (5 minutes 37° C.) and imaged by fluorescence microscopy using a Nikon A1 camera (Nikon) linked to a Nikon Eclipse Ti2-E Inverted Microscope Imaging System (Nikon) for each channel. Fifteen images per well were acquired.

### Colony Formation Assay

10³ PDAC cells were plated in 2.5 ml of media on 6-well plates with or without 10⁵ CAFs in DMEM supplemented with 10% v/v FBS and 2 mM L-glutamine with 100 μg/ml Penicillin/Streptomycin. In 24 hours, the old media was replaced with new media according to experimental conditions. After 10 days, the cultures were washed with PBS twice and fixed in 10% methanol/10% acetic acid solution for 15 minutes at RT and stained for 20 minutes with 0.4% Crystal Violet in 20% ethanol. Plates with colonies were scanned and images were analyzed with ImageJ software using the Colony Area plugin (Guzman et al., PLoS One, 2014, 9, e92444).

### Annexin V Apoptosis and Viability Assays

PDAC cells were pre-labeled with eFluor670 (CPD670) according to the manufacturer's instructions (#65-0840-85, eBioscience) and plated either alone or in co-cultures with CAFs at 10⁵ cells in 2 ml per well in 6-well plates in DMEM supplemented with 10% FBS, 2 mM L-glutamine, and 100 μg/ml Penicillin/Streptomycin. In 24 hours, the medium was replaced with indicated experimental media supplemented with 10% FBS, 10% lipid-depleted serum (LDS), or LDS supplemented with purified donor LDL (#360-10, LEE Biosolutions, Maryland Heights, MO, USA). After 72 hours, cells were harvested by trypsinization and stained with Annexin V-FITC (#640906, Biolegend) or Annexin V-PE (#640908, Biolegend) according to the manufacturer's instructions. Dead cells were determined by DAPI staining (#D9542, Sigma-Aldrich), fluorescence was measured using BD LSRII flow cytometer and analyzed with FlowJo software.

For direct enumeration of viable cells in co-cultures with CAFs, we seeded CAFs at 12,000 cells per well in 96-well plate (#3603, Corning) in DMEM/10% FBS. The next day, the media was replaced with 10%/LDS/DMEM and 4,000 DsRed-tagged Panc-1 cells were plated on the top of CAFs at 3:1 ratio. After 96 hours, fluorescence of viable DsRed-positive Panc-1 cells was measured using Spark Multimode Microplate Reader (Tecan) with parameters set at 540 nm for excitation and 590 nm for detection. To measure the dynamics of cell proliferation in vitro, xCELLigence E-Plate (ACEA BioSciences) was used as per the manufacturer's recommended protocol. Impedance values were recorded every hour. Data was graphed using GraphPad Prism software.

### Trogocytosis Assays In Vitro by Flow Cytometry

To label cellular membranes, pairs of lipophilic fluorescent dyes were used according to the manufacturer's instructions in the following combinations: 1) adherent CAFs were stained 1 μM PKH67 (Sigma-Aldrich), Panc-1 were stained in suspension with 10 μM CellTracker™ orange CMTMR (Thermo Fisher Scientific); 2) CAFs labeled with 0.5 μM BODIPY-cholesterol (Topfluor Cholesterol, #DO-016545, Avanti Polar Lipids) dissolved in 0.1 mM methyl-b-cyclodextrin (#C4555, Sigma) with 2 μM cholesterol (#C8667, Sigma-Aldrich) in MEM media at 37° C. for 30 minutes in suspension; murine or human (Panc-1) PDAC cells were stained in suspension with CPD670 (#65-0840-85, Thermo Fisher Scientific). After labeling, CAFs were co-incubated for indicated time intervals with PDAC cells in 12-well culture plates at ratio of 2:1 in half DMEM media. Co-cultures of CAFs and PDAC cells for 3 minutes on ice were used as negative controls. In experiments assessing the effect of extracellular calcium on trogocytoisis, cells were co-cultured in Phosphate Buffered Saline (PBS) containing calcium and magnesium (D8662, Sigma-Aldrich) or in PBS without calcium and magnesium (D8537, Sigma-Aldrich). After co-cultures, cells were collected and pelleted by centrifugation (5 minutes, 1500 rpm), resuspended in 10% formalin (Sigma) for 10 minutes at RT, washed and re-suspended in PBS/1% BSA. Trogocytosis was measured as the acquisition of CAF-specific fluorescence in PDAC cells by flow cytometry on MACSQuant® VYB Flow Cytometer (Miltenyi Biotec) or FACScan analyzer (BD Biosciences). Analysis of dual staining was done using FlowJo software (10.8.1, BD Biosciences). Live cells were gated based on FSC/SSC parameters, then single cells were gated based on SSC-A/SSC-H.

For interference with trogocytosis, CAFs were pre-treated prior to co-cultures for 2 hours with recombinant label-free 100 nM Annexin-V (#ab157342, Abcam) or 100 nM Zn-DPA (Molecular Targeting Technologies) and subsequently maintained in co-cultures at the same concentrations.

For trogocytosis between OT-1 and CAF cells, T cells were isolated from splenocytes of OT1 mice (strain 021880, The Jackson Laboratory) and stimulated with OVA₂₅₇₋₂₆₄ (1 μg/ml) for 24 hours. CTLs were labeled with DID (DiIC18(5); 1,1′-dioctadecyl-3,3,3′,3′-tetramethylindodicarbocyanine, 4-chlorobenzenesulfonate salt, D7757, Invitrogen) and then co-cultured with CAFs at 5:1 ratio in the presence of 0.05% DMSO as diluent or indicated agents: niclosamide (1 μM), clofazimine (1 μM), anti-TIM3 Ab (4 μg/ml, clone RMT-3-23, BioXCell, Lebanon, NH), or 25-hydroxycholesterol (4 μM, #H1015, Sigma-Aldrich) for 12 hours followed by flow cytometry analysis.

### Analysis of Cellular Lipids Using LC-MS.

Optima grade methanol, water, acetonitrile, methyl tert-butyl ether, and 2-propanol were from Thermo Fisher Scientific (Pittsburgh, PA). Gasses were supplied by Airgas (Philadelphia, PA). Glassware and HPLC vials were from Waters Corp (Milford, MA).

Lipid extraction: Frozen cell pellets (˜1×10⁶ cells) were collected in low retention Eppendorf tube and mixed with 0.6 mL 80% methanol (MeOH) and 20 μL of lipidomics internal standard mix (1:1, SPLASH® LIPIDOMIX #330707: Ceramide/Sphingoid Internal Standard Mixture I #LM6002, both from Avanti Polar Lipids, Alablaster, AL) and kept in dry ice. Samples were pulse sonicated for 30× half-second pulse on ice and kept on ice for 20 minutes for metabolites extraction. Each tube was then vortexed 3×30 seconds each. The cell homogenates were moved to a 10 mL glass Pyrex tube with screw cap. The Eppendorf tubes were rinsed with 0.5 mL methanol and added to the same glass tube. 5 mL methyl tert-butyl ether (MTBE) was added to each of the tubes and then tubes were shaken vigorously for 30 minutes. 1.2 mL water was added to each tube and vortexed for 30 seconds each. Centrifugation for 10 minutes at 1000×g created two phases. The top clear phase was moved to a clean glass Pyrex tube and dried down under nitrogen. 200 μL MTBE/MeOH=1/3 (v/v) per 10 mg tissue was used to re-suspend the residue. The sample was spun down at 10,000×g for 10 minutes at 4° C. and only 100 μL were transferred to a HPLC vial for LC-MS analysis. A pooled sample was created by mixing 20 μL of each re-suspended sample and ran as quality control (QC) every 15 samples. 2 μL injections were made in both positive mode and separately in the negative mode.

Liquid chromatography high resolution-mass spectrometry (LC-HRMS) for lipids: Lipid analysis on the extracted samples was conducted by liquid chromatography-high resolution mass spectrometry as previously described (Wang et al., J. Lipid Res., 2022, 63, 100255). Briefly, separations were conducted on an Ultimate 3000 (Thermo Fisher Scientific) using an Ascentis Express C18, 2.1×150 mm 2.7 μm column (Sigma-Aldrich, St. Louis, MO). The flow-rate was 0.4 m/min, solvent A was water:acetonitrile (4:6 v/v) with 0.1% formic acid and 10 mM ammonium formate and solvent B was acetonitrile:isopropanol (1:9 v/v) with 0.1% formic acid and 10 mM ammonium formate. The gradient was as follows: 10% B at 0 minutes, 10% B at 1 minute, 40% B at 4 minutes, 75% B at 12 minutes, 99% B at 21 minutes, 99% B at 24 minutes, 10% B at 24.5 minutes, 10% at 30 minutes. Separations were performed at 55° C.

For the HRMS analysis, a QE Exactive-HF mass spectrometer (Thermo Fisher Scientific) calibrated within 3 days was used in positive ion mode with a HESI source. The operating conditions were: spray voltage at 3.5 kV; capillary temperature at 285° C.; auxiliary temperature 370° C.; tube lens 45. Nitrogen was used as the sheath gas at 45 units, the auxiliary gas at 10 units and sweep gas was 2 units. Same MS conditions were used in negative ionization mode, but with a spray voltage at 3.2 kV. Control extraction blanks were made in the same way using just the solvents instead of the tissue homogenate. The control blanks were used for the exclusion list with a threshold feature intensity set at 1e10^{\hat{ }5}. Untargeted analysis and targeted peak integration was conducted using LipidsSearch 4.2 (Thermo Fisher Scientific) as described (Wang et al., Bioanalysis, 2021, 13, 1037-1049). All samples were analyzed in a randomized order in full scan MS that alternated with MS2 of top 20, with HCD scans at 30, 45 or 60 eV. Full scan resolution was set to 120,000 in the scan range between m z 250-1800. The pool sample was run every 15 samples. Lipids quantification was done from the full scan data. The areas were normalized based on the amount of the internal standard added for each class (as found in FIG. 12). All amounts were normalized to the original tissue weight.

Data analysis was done in MetaboAnalyst 5.0, where the raw peak intensities associated with each metabolite were analyzed using one factor statistical analysis. The processed data were organized into groups that were compared and graphed onto volcano plots to show significantly upregulated and downregulated lipid species.

Analyses of ANO6 mRNA and Protein Expression in Human Cancers

Data for mRNA expression and patient outcomes for human pancreatic ductal adenocarcinoma, cervical cancer and breast cancer were extracted from the publicly available NCI TCGA program. ANO6 protein expression data were obtained from the NCI Clinical Proteomic Tumor Analysis Consortium (CPTAC) dataset. Analyses to compare the overall survival among the ANO6-high and ANO6-low were performed by comparing the Kaplan-Meyer survival curves with log-rank tests. These were calculated using the R ‘survival’ package (Themeau et al., Modeling survival data: extending the Cox model. Statistics for biology and health, 2000, New York, Springer, xiii, p. 350).

### Quantification and Statistical Analysis

For analysis of continuous data, Wilcoxon tests, Mann-Whitney and Student t-test were used as indicated, and binary outcomes were compared using Fisher's exact test. Repeated measures (i.e., multiple measures within a single mouse) were analyzed using generalized linear regression models with Generalized Estimating Equations (GEE). Growth curves were modeled using linear regression with interactions between treatment and time, again using GEE to account for within-sample correlation. Survival time outcomes were assessed using Kaplan-Meier curves with log-rank tests. The statistical details of experiments can be found in the figure legends, figures and text of the Results.

### Preparation of Fibroblast-Derived 3D Matrices

Cell derived matrix was obtained using published protocols (Franco-Barraza et al., Elife, 2017, 6). Confluent fibroblastic cultures were maintained for 5 days in the presence of daily added and freshly prepared ascorbic acid at a concentration of 50 μg/ml. The resulting ‘unextracted’ 5 day long multilayered 3D cultures were processed for phenotypic characterization and ECM fiber alignment. Fibronectin channel monochromatic images were analyzed via ImageJ's (SCR 003070) OrientationJ plugin (SCR 014796) as described previously (Rezakhaniha et al., Biomech. Model Mechanobiol, 2012, 11, 461-473). Numerical outputs were normalized by setting mode angles to 0°, and correcting angle spreads to fluctuate between −90 and 90. Angle spreads for each experimental condition, corresponding to a minimum of three experimental repetitions and five image acquisitions per condition were plotted and their standard deviations calculated using Excel spreadsheets. Percentage of fibers oriented −15 and 15 was determined for each normalized image-obtained data.

### Example 2: Intracellular Transfer of Free Cholesterol from Stroma to PDAC

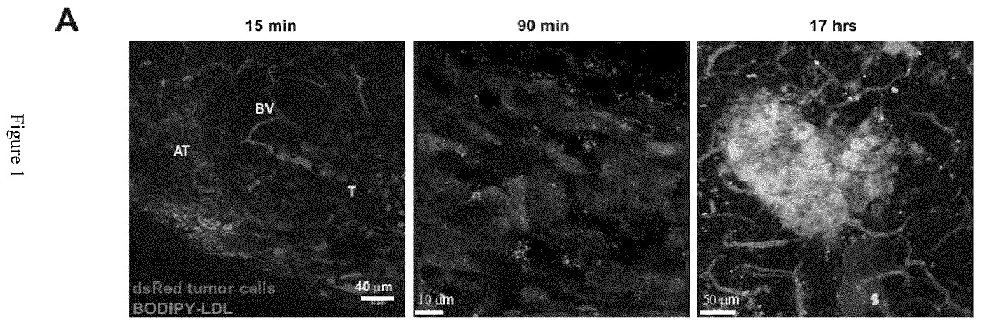

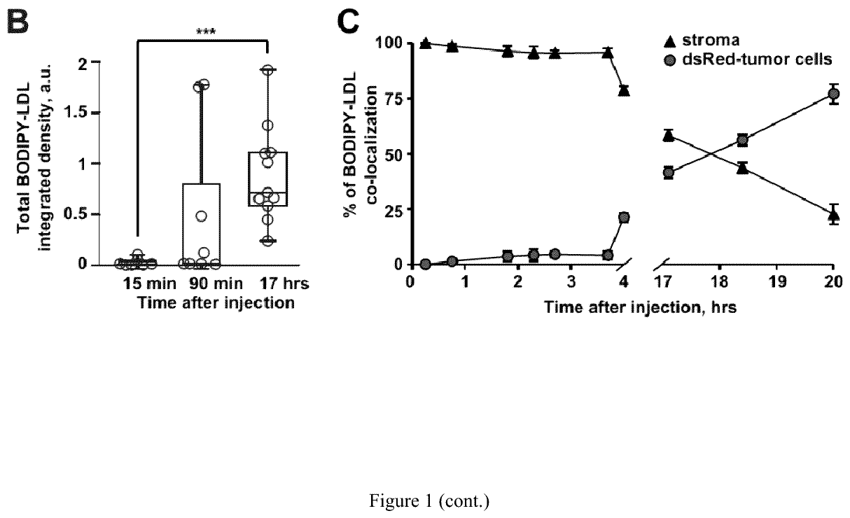

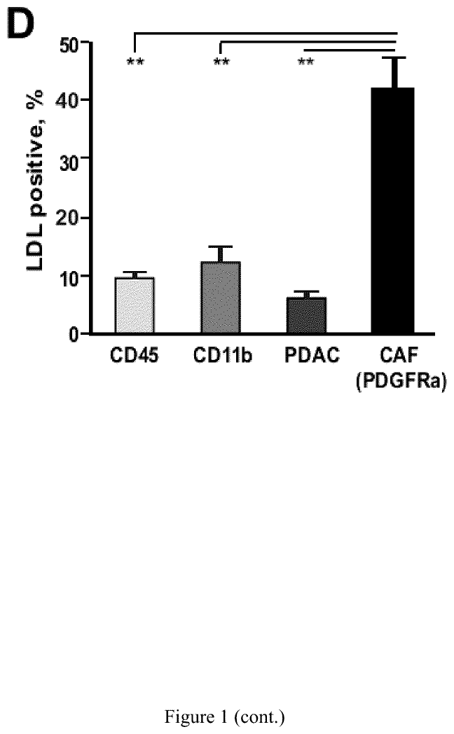

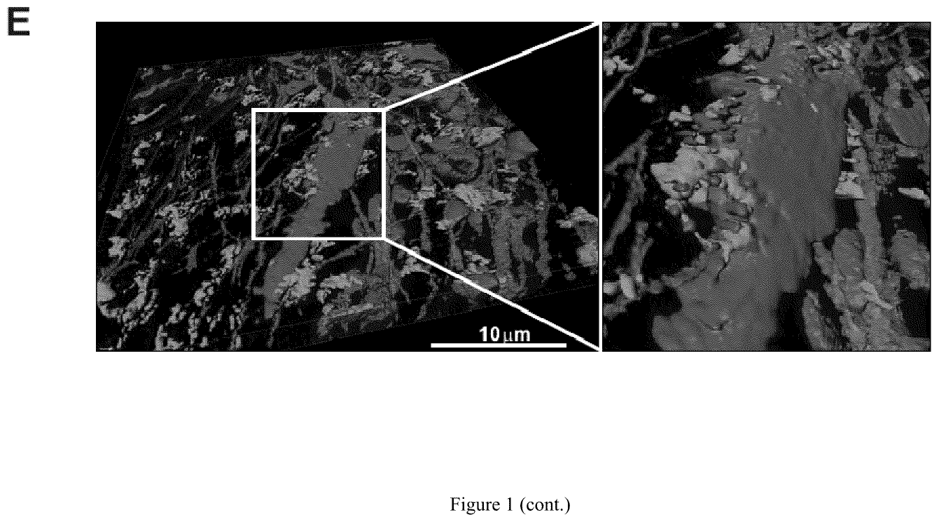

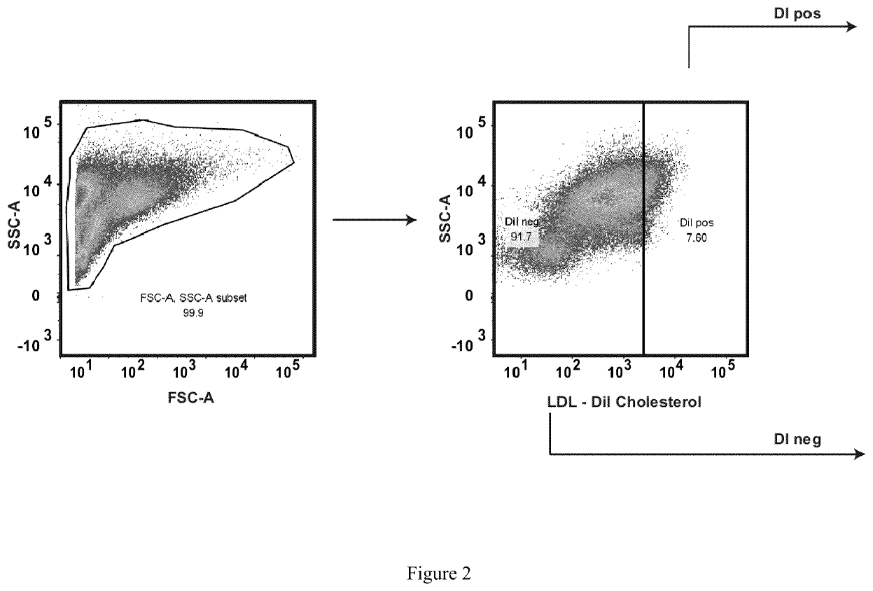

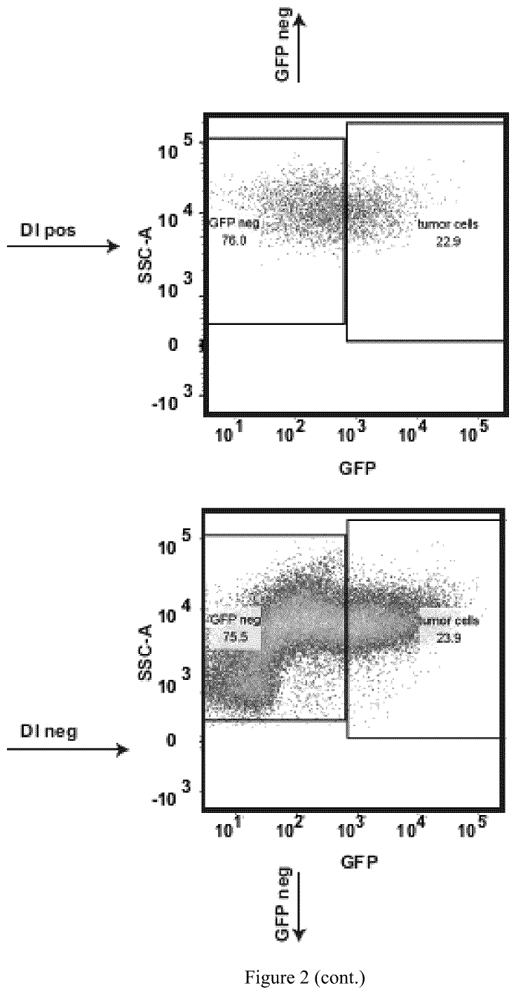

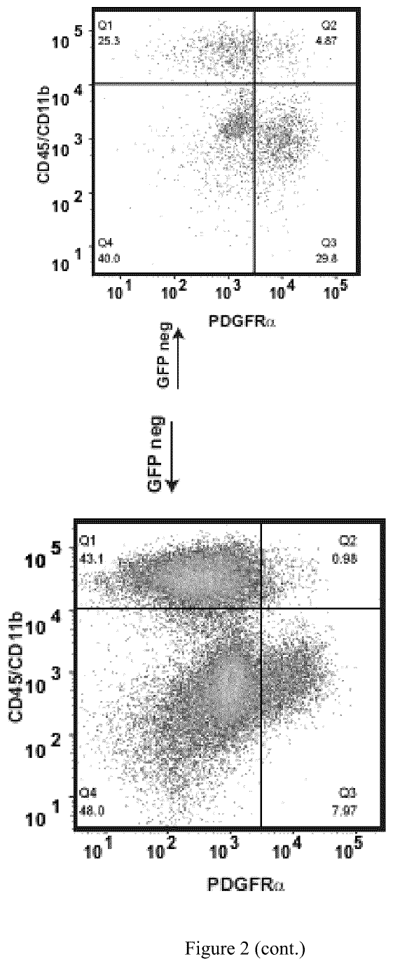

Pancreatic cancer stroma as a transit station for lipids delivery. To gain insight into the ability of PDAC cells to take up exogenous cholesterol in vivo, the time-course and the transit dynamics of blood-borne low-density lipoprotein (LDL) to cancer cells within the pancreatic tumor mass generated was determined by orthotopic implantation of DsRed-labeled syngeneic murine PDAC cells deficient in Nsdhl, an essential cholesterol pathway gene (FIG. 1, Panel A). Using multiphoton intravital microscopy of these orthotopic pancreatic tumors, the rate of cancer cell uptake of intravenously injected LDL particles containing relatively inert BODIPY-conjugated cholesterol (green) was traced. As the LDL particles (25 nm) rapidly enter the blood vessels and permeate to the interstitial fluid, the uptake of LDL by the cells within the pancreatic tumor mass becomes visible as large intracellular endosomes containing BODIPY-cholesterol (FIG. 1, Panel A and Panel B). A more extensive time course, comparing BODIPY signal within DsRed-positive PDAC cells versus unlabeled stroma, was performed. Unexpectedly, for up to 4 hours post-injection the exogenously delivered lipids localized exclusively to the stroma, and not to the cancer cells (FIG. 1, Panel C). This was confirmed by FACS analysis at 90 minutes post-injection that LDL was predominantly observed in PDGFRα-positive cancer-associated fibroblasts (CAFs), but minimally in fluorescently tagged PDAC cells, or in CD11b-positive, or CD45-positive immune cells (FIG. 1, Panel D, FIG. 2). Following this initial delay, BODIPY-cholesterol progressively co-localized to the DsRed-positive pancreatic carcinoma cells at 17-20 hours post intravenous injection (FIG. 1, Panel C), whereas the amount of BODIPY-cholesterol in the stroma diminished to the background level. Most of the BODIPY-positive PDAC cells were in direct physical contact with BODIPY-positive stromal cells with clearly visible interdigitations of their plasma membranes (PM) (FIG. 1, Panel E). These results implied that exogenous LDL lipids predominantly transit via CAFs for delivery to pancreatic cancer cells.

### Example 3: Cells Need Direct Contact for the Efficient Transfer of Cholesterol to PDAC Cells

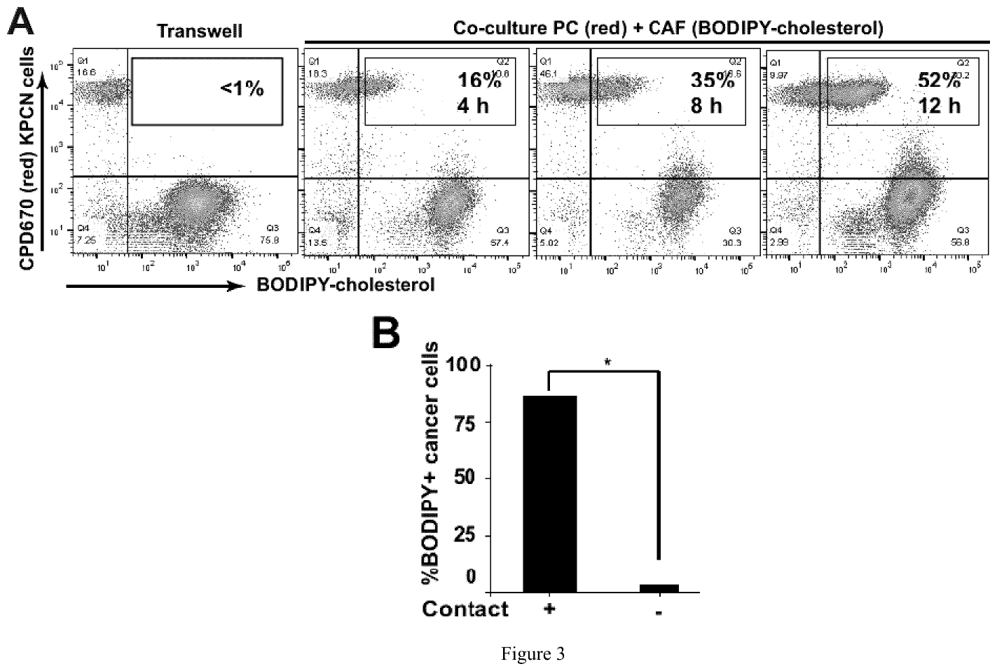

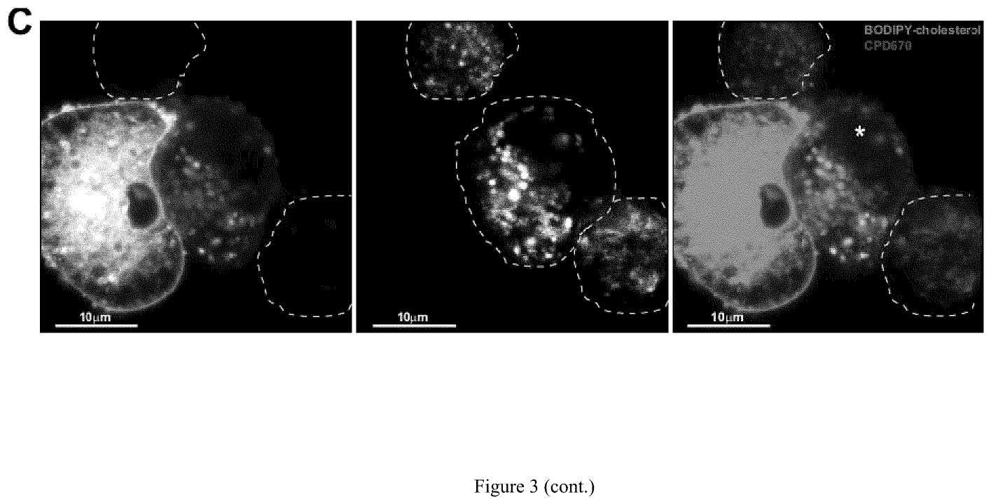

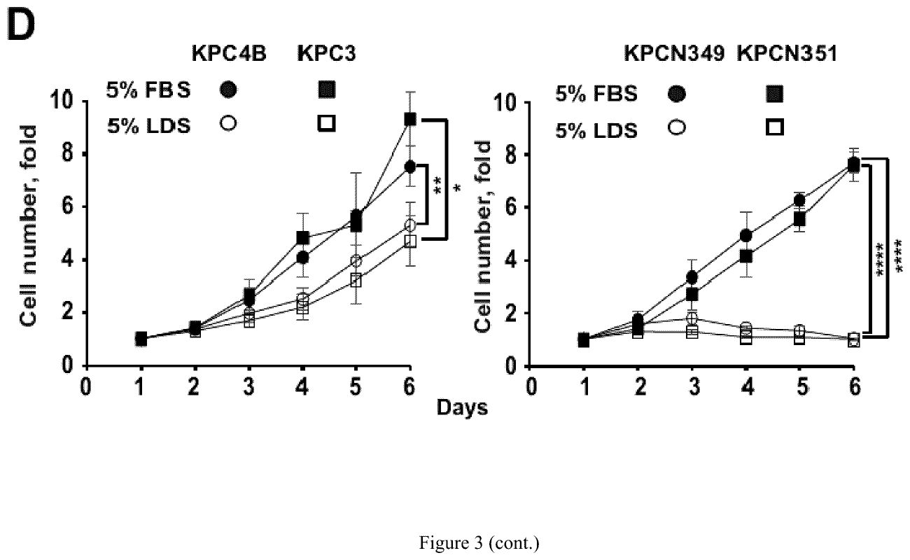

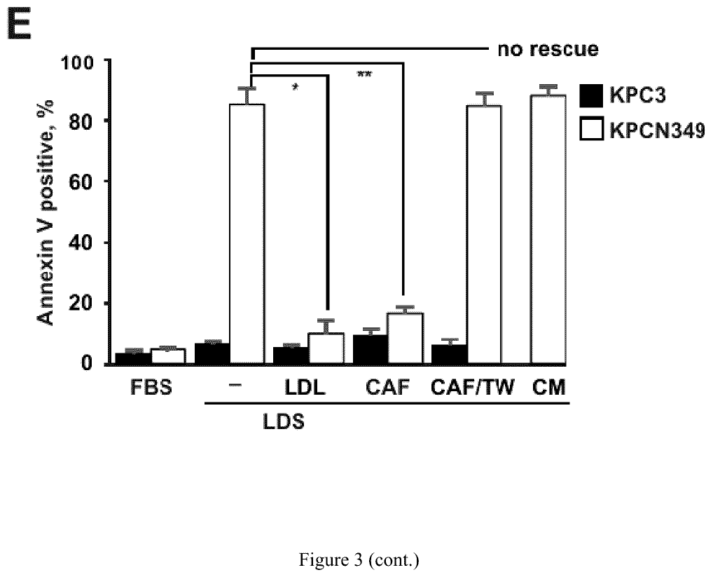

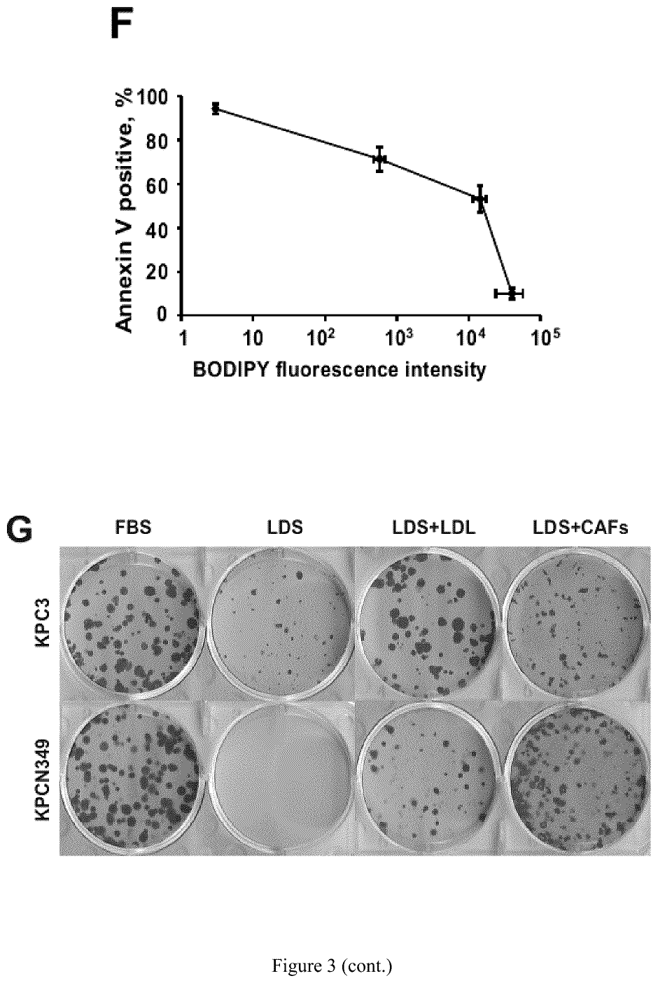

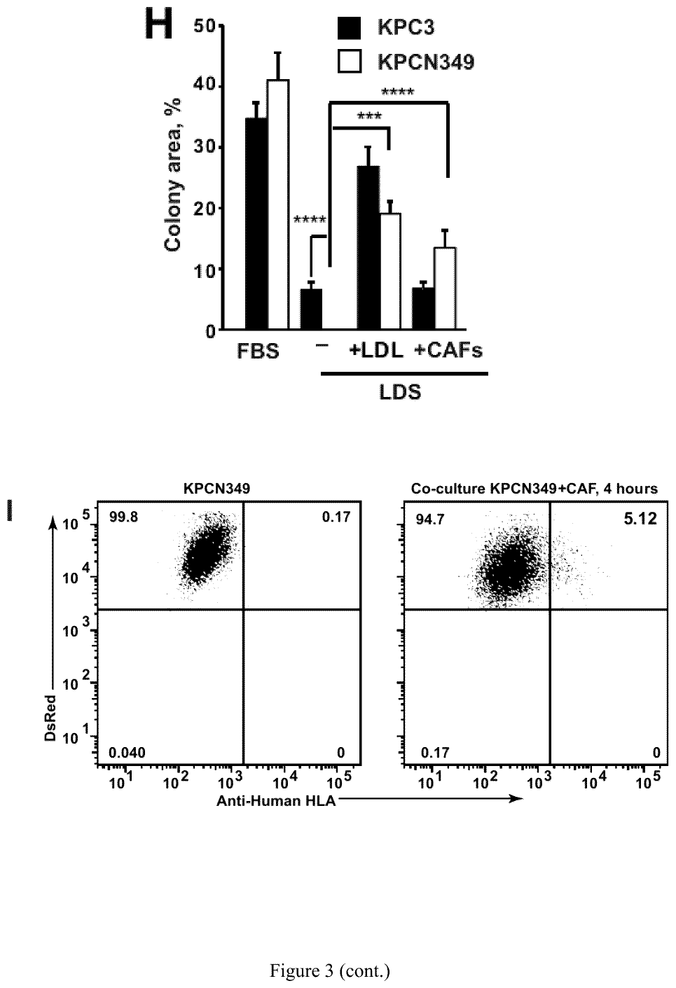

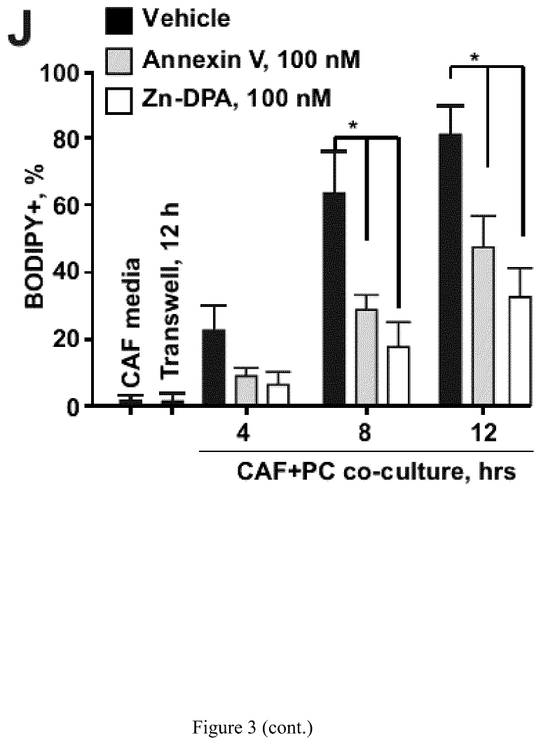

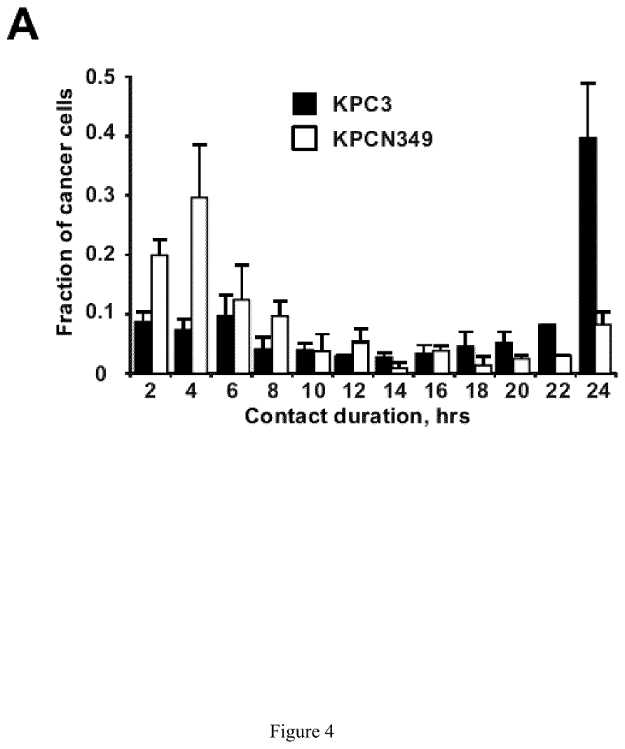

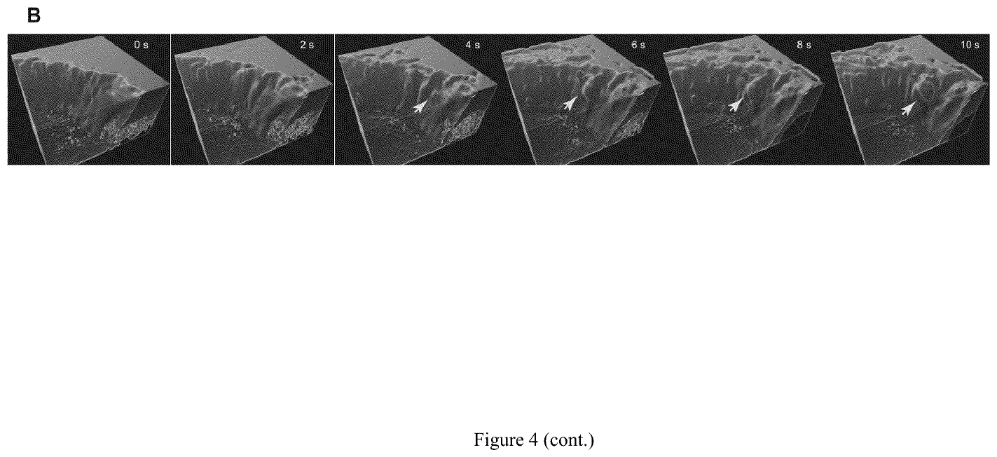

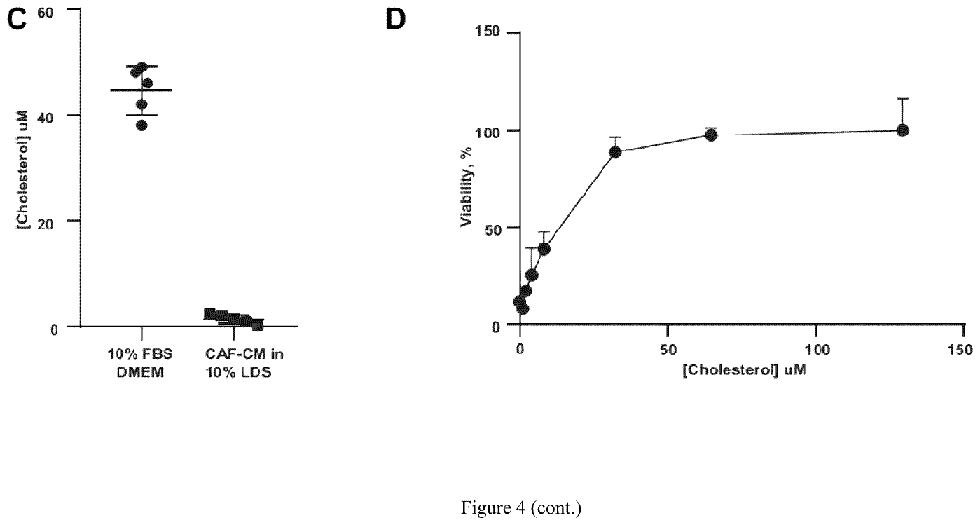

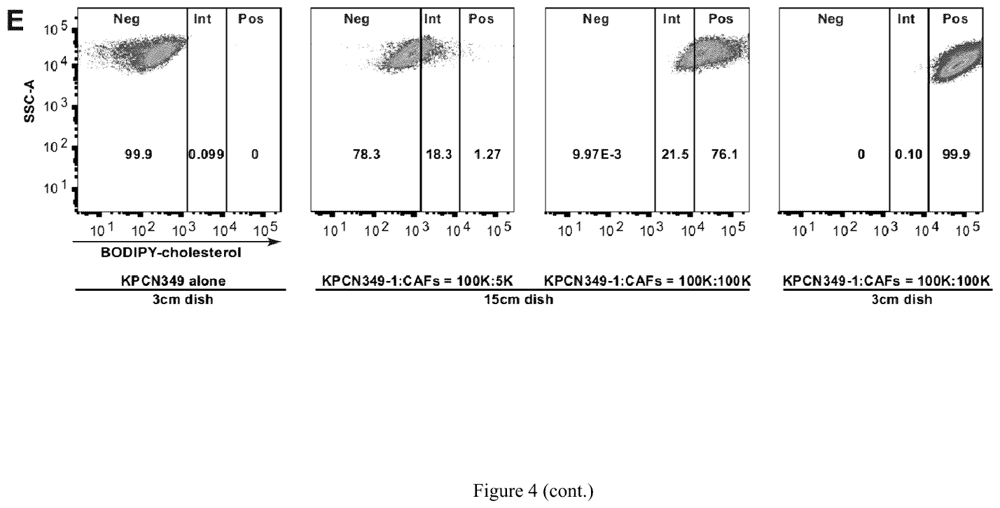

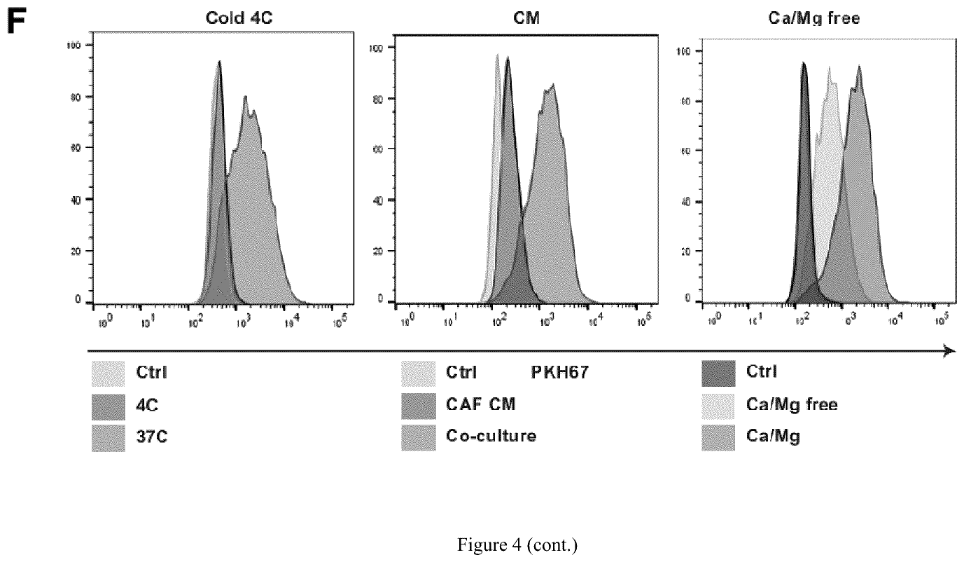

CAFs provide cholesterol to PDAC cells via a cell contact-dependent mechanism. To determine whether the LDL-cholesterol transfer observed in vivo occurred in vitro, BODIPY-cholesterol labeled CAFs were co-cultured with PDAC cells. This resulted in a rapid (4-12 hours) acquisition of BODIPY signal by PDAC cells (FIG. 3, Panel A). Importantly, visual enumeration revealed that ability to acquire BODIPY-cholesterol was largely limited to those PDAC cells, which were in direct physical contact with CAFs (FIG. 3, Panel B and Panel C, and FIG. 4, Panel A), as no transfer was observed between cells separated by a Transwell membrane. Detailed microscopic analyses of time-lapse images of PDAC cells demonstrated them making synapse-like contacts with CAFs (FIG. 3, Panel C, and FIG. 4, Panel B), and showed that PDAC cells acquire bleb-like protrusions from the CAF PM at the site of cell contacts.

To investigate further the functional significance of the in vivo transfer of LDL-borne BODIPY-labeled cholesterol to PDAC cells, the ability of CAFs to support the viability of cholesterol-auxotrophic PDAC cells in vitro was investigated. In the absence of lipids and cholesterol in the media (growth in lipid depleted serum, or LDS), KPC cell lines with an intact Nsdhl gene had only minor growth delay in LDS medium, and did not undergo apoptosis (FIG. 3, Panel D and Panel E). In contrast, the Nsdhl-null KPCN349 and KPCN351 cells did not grow, as anticipated due to the cholesterol auxotrophy imposed by Cre-mediated deletion of this essential cholesterol pathway gene (FIG. 3, Panel D), and these cells underwent apoptosis after 72 hours (FIG. 3, Panel E). However, apoptosis was completely rescued by direct addition of LDL particles delivering 50 μM cholesterol, or by co-culture with human CAFs derived from PDAC patients similarly prevented apoptosis (FIG. 3, Panel E). However, cell contact was essential for this ability of CAFs, as conditioned medium (CM), or co-culture with CAFs separated from PDAC cells by a mesh in Transwell plates (4 μm pores permeable for soluble molecules and lipid particles) failed to rescue the LDS-cultured Nsdhl-null PDAC cells (FIG. 2, Panel E). Even though the CAF-conditioned media contained measurable concentrations of cholesterol (3-5 μM, FIG. 4, Panel C), such level of cholesterol was insufficient to support the viability of the cholesterol-auxotroph PDAC cells (which required greater than 30 μM LDL cholesterol to maintain PDAC cells viability in vitro, FIG. 4, Panel D). The effect of cell plating density on the rate of BODIPY-cholesterol uptake by KPCN349 cells in 24 hours of co-culture with CAFs was examined (FIG. 4, Panel E). Furthermore, the likelihood of PDAC-CAF cell contacts was varied by altering the CAF-PDAC cell ratio and plating cell density and an inverse relationship was found between the uptake of BODIPY-cholesterol in CAF membranes and the percentage of annexin-V positive apoptotic PDAC cells (FIG. 3, Panel F).

To investigate the durability of PDAC dependence on CAFs, long-term cultures of KPCN and KPC cancer cells were maintained in FBS, LDS, LDL+LDL, and LDS+CAF conditions (FIG. 3, Panel G and Panel H). Clonogenic analysis indicated that CAFs can provide ample supply of cholesterol to maintain PDAC cell viability and long-term growth. PDAC cells obtain membrane cholesterol via trogocytosis of CAF membranes. The process of direct transfer of nutrients via cell contact has been previously described in the immune cells (Ritter et al., J. Vis. Exp., 2018, 141; and Yang et al., Cell, 2012, 151, 111-22) and in development (Whitlock et al., Annu. Rev. Physiol., 2017, 79, 119-143) as trogocytosis. Trogocytosis is a process by which the cells engaged in direct physical contact acquire protrusions of PM. In CTLs, trogocytosis causes uptake and expression of the human leukocyte antigen (HLA) MHC-I and peptide complexes taken up from the antigen-presenting cells (Ritter et al., J. Vis. Exp., 2018, 141; Yang et al., Cell, 2012, 151, 111-22; and Suzuki et al., Science, 2013, 341, 403-406). Importantly, upon co-culture of human patient-derived CAFs with mouse PDAC cells, a similar acquisition and re-expression by murine PDAC of human HLA proteins on the surface was observed (FIG. 3, Panel I).

Exposure of phosphatidylserine (PtdSer) on the outer leaflet of the PM has been established as the “eat me” signal in trogocytosis executed by T cells (Suzuki et al., Nature, 2010, 468, 834-838; and Suzuki et al., J. Biol. Chem., 2013, 288, 13305-13316). It was next tested if blockade of PtdSer antagonizes CAF membranes trogocytosis by PDAC cells. Indeed, masking PtdSer on the surface of CAFs by incubation in the presence of label-free annexin V or with zinc-dipicolylamine (Zn-DPA) (Peng et al., Cell. Res., 2019, 29, 725-738) significantly reduced the uptake of BODIPY-labeled CAF membranes (FIG. 3, Panel J) indicating that PtdSer externalization is important for trogocytosis at the CAF-cancer cell contacts. Notably, trogocytosis of the CAF membranes was markedly reduced in calcium-free media, or at +4° C. (FIG. 4, Panel F). Taken together, these results indicate CAF trogocytosis requires calcium-dependent PtdSer movement from the inner to the outer leaflet of the CAF PM.

### Example 4: CAFs Lacking TMEM16F are the CAFs Least Able to Support Cancer Survival in the Absence of Exogenous Lipids

ANO6 scramblase confers poor survival in pancreatic cancer. To gain mechanistic insight into regulation of trogocytosis in PDAC, the candidate enzymes responsible for PtdSer externalization in CAFs was sought. Exposure of PtdSer on the outer leaflet of PM is the consequence of lipid redistribution, or “scrambling”, between the inner and outer leaflets of the PM to be regulated by lipid scramblases (Yang et al., Cell, 2012, 151, 111-122; and Whitlock et al., Annu. Rev. Physiol., 2017, 79, 119-143). In contrast to Xk-related 8 (XKR8) scramblase involved in the exposure of PtdSer on the external leaflet of PM during apoptosis (Suzuki et al., Science, 2013, 341, 403-406; and Suzuki et al., Nature, 2010, 468, 834-838), in non-apoptotic cells phospholipid scramblase anoctamin 6 (ANO6) induces PtdSer externalization in response to elevated intracellular calcium under physiological stimulation (Yang et al., Cell, 2012, 151, 111-122; and Suzuki et al., Nature, 2010, 468, 834-838). In platelets, ANO6 is required for platelet aggregation and the release of extracellular vesicles (Suzuki et al., Nature, 2010, 468, 834-838; and Suzuki et al., J. Biol. Chem., 2013, 288, 13305-13316), and mutations in ANO6 result in a bleeding disorder in humans known as Scott's syndrome (Suzuki et al., Nature, 2010, 468, 834-838). Since ANO6 has not been previously implicated in cancer, the expression of anoctamin paralogs in human and mouse (Peng et al., Cell Res., 2019, 29, 725-738) and mouse (Gabitova-Cornell et al., Cancer Cell, 2020, 38, 567-583) pancreatic cancer in single-cell RNA sequencing datasets was examined.

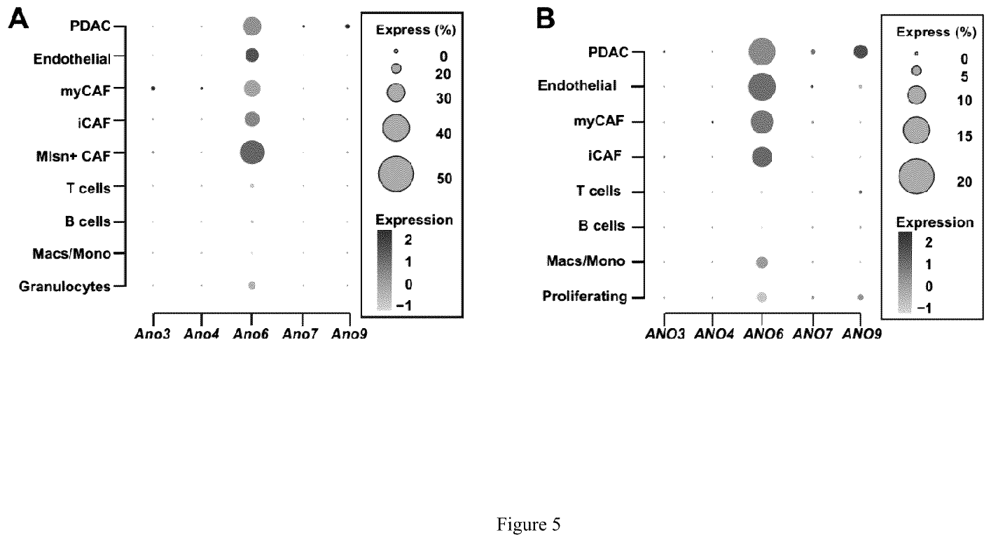

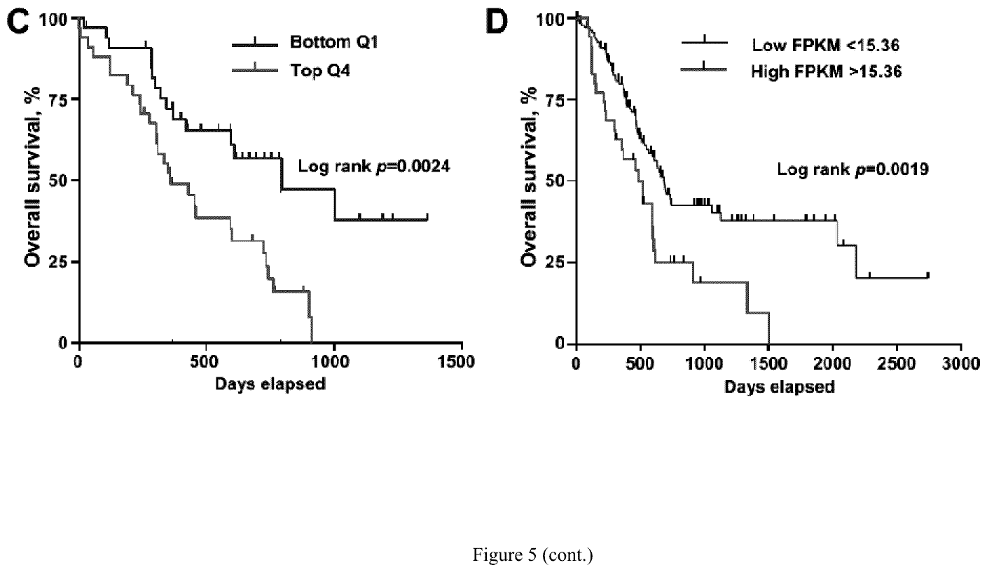

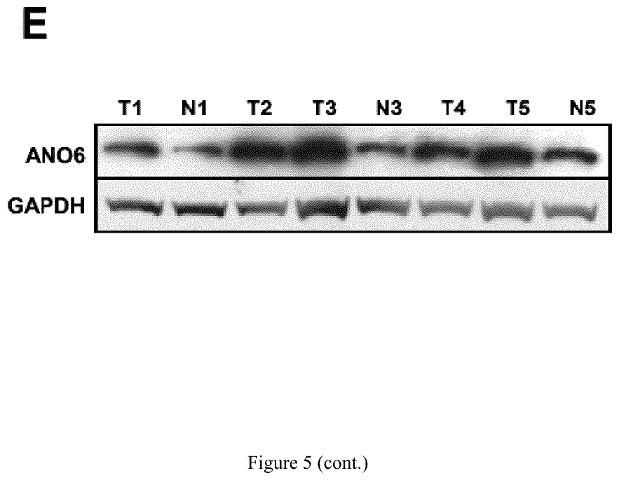

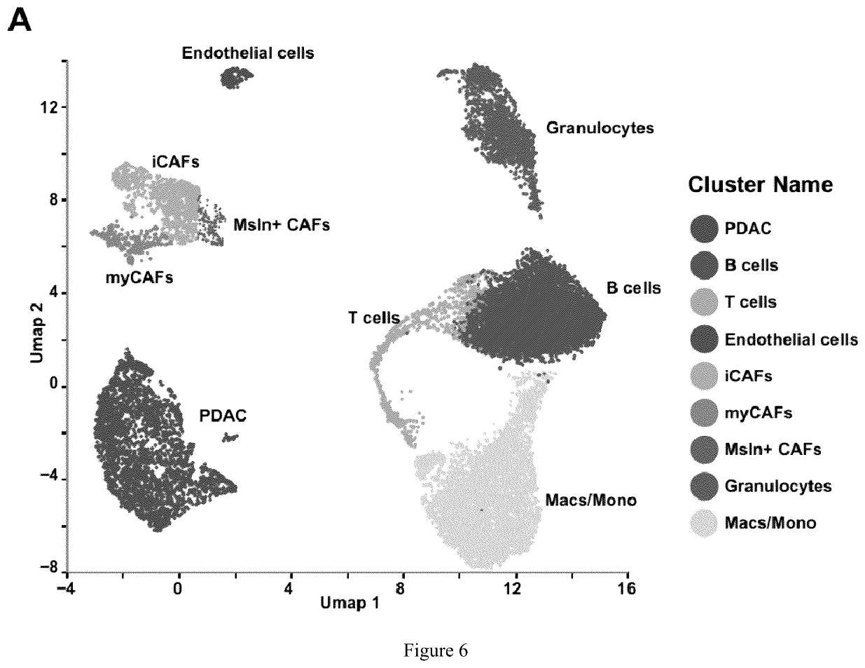

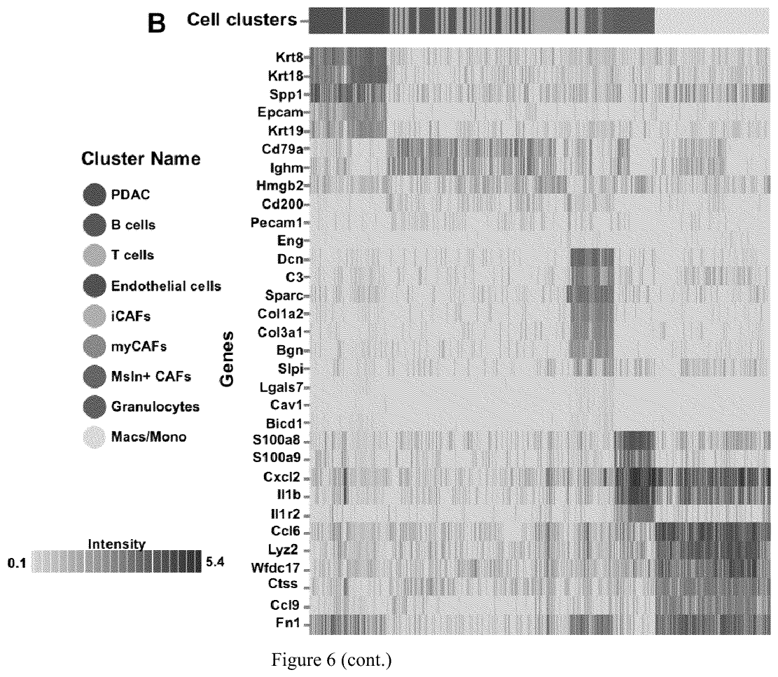

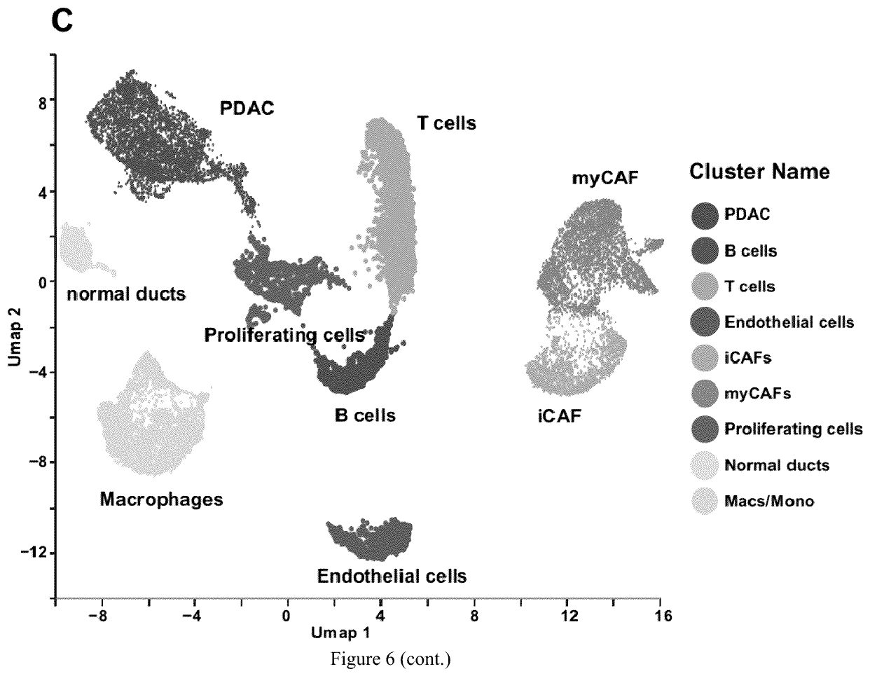

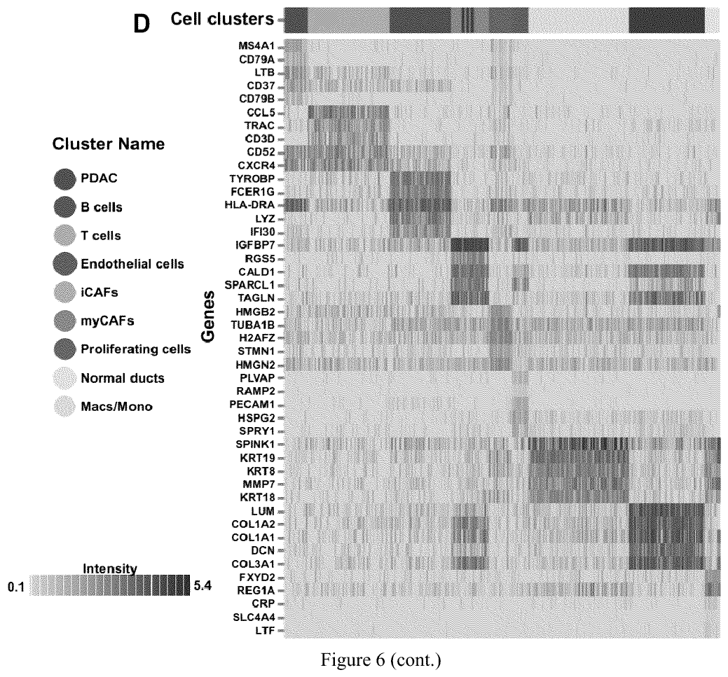

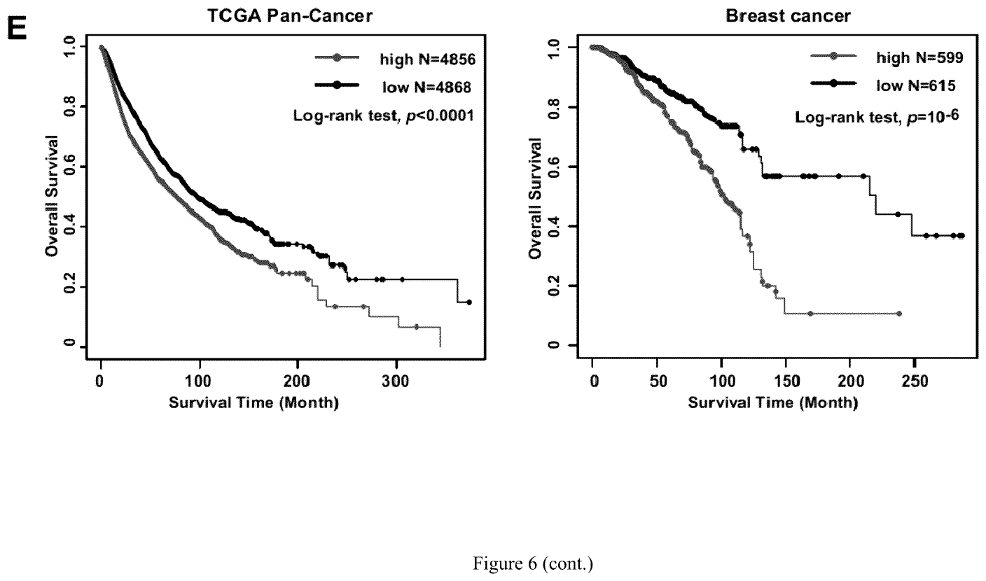

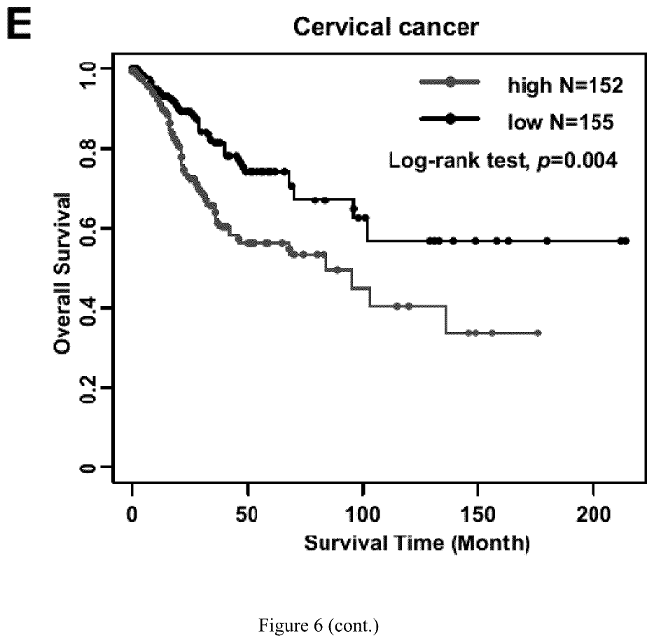

Of the five Ano6 paralogs in mice (Ano3, 4, 6, 7 and 9) with experimentally confirmed PtdSer scramblase activity (Suzuki et al., J. Biol. Chem., 2013, 288, 13305-13316) only Ano6 was expressed in CAFs, endothelial cells and adenocarcinoma cells (FIG. 5, Panel A, and FIG. 6, Panel A and Panel B) within the KPC tumor cellular population. The pattern of ANO6 expression in human PDAC was similar suggesting a unique and non-redundant function of this scramblase (FIG. 5, Panel B, and FIG. 6, Panel C and Panel D). Furthermore, high expression of ANO6 protein (FIG. 5, Panel C) or transcript (FIG. 5, Panel D) are associated with worse overall survival of patients with stages 1-3 of PDAC as well as in breast and cervical cancers in the TCGA cohort (FIG. 6, Panel E). The expression of ANO6 by western blot on a panel of fibroblastic cells obtained from surgical pancreatic adenocarcinoma tissues was also assessed (FIG. 5, Panel E). There was consistently higher expression of ANO6 in fibroblastic cells derived from the tumor compared to those from adjacent non-malignant human pancreatic tissues. Together, these results support a pro-tumoral role for ANO6 in PDAC and other human cancers.

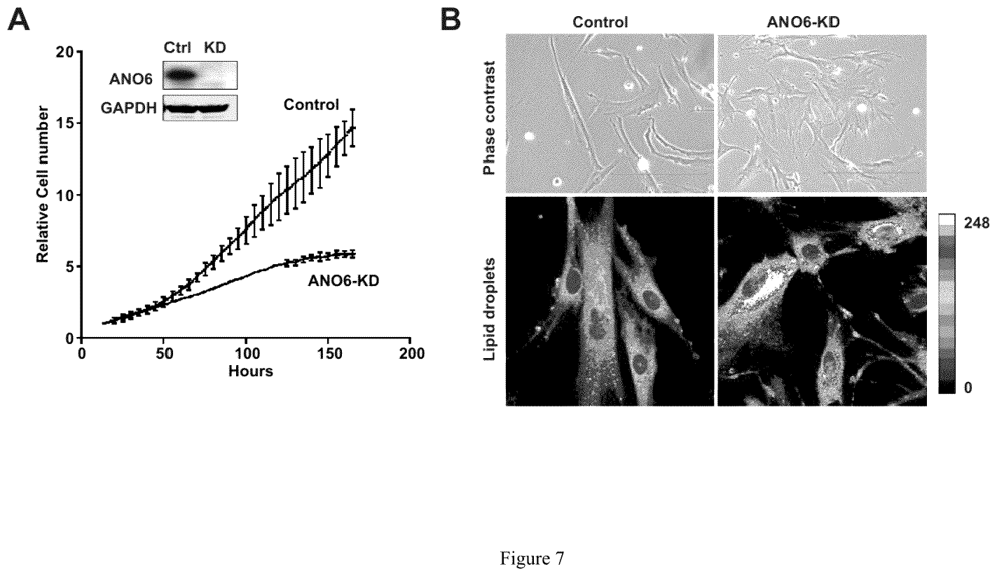

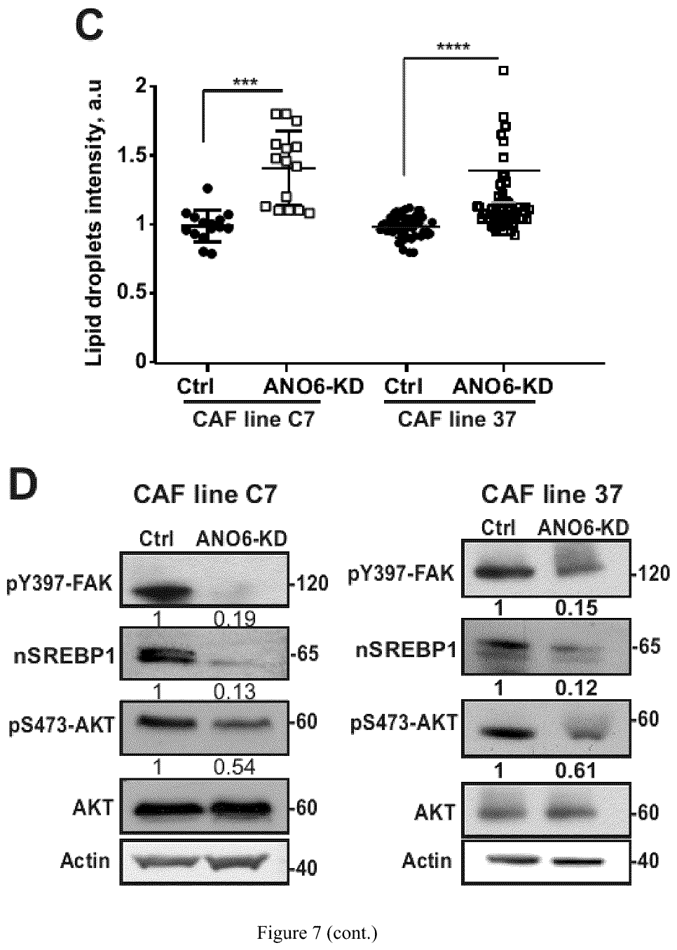

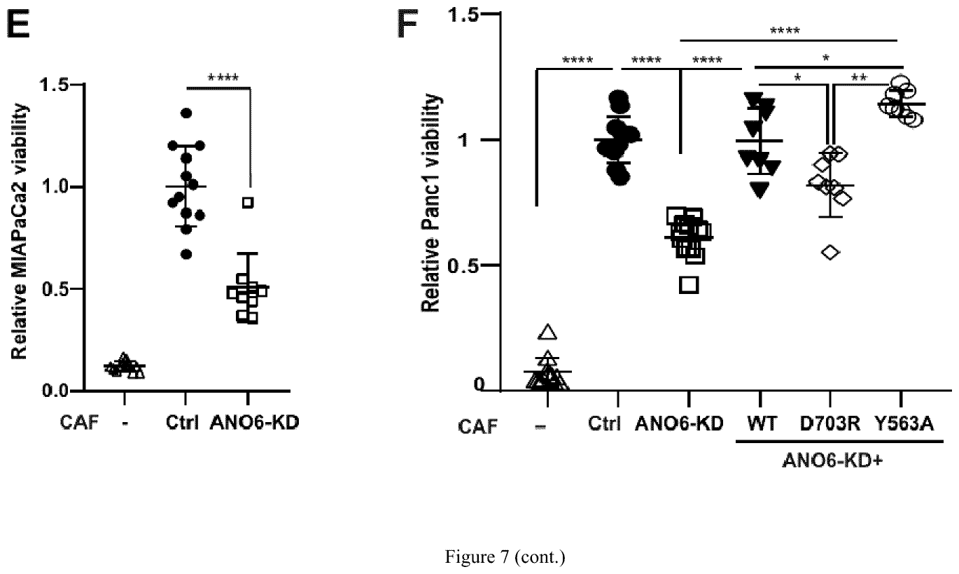

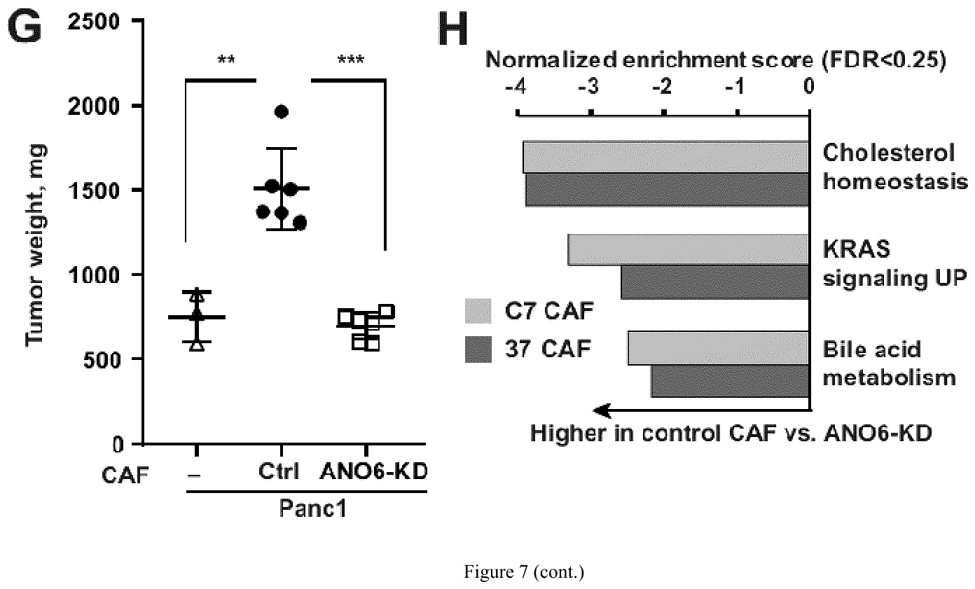

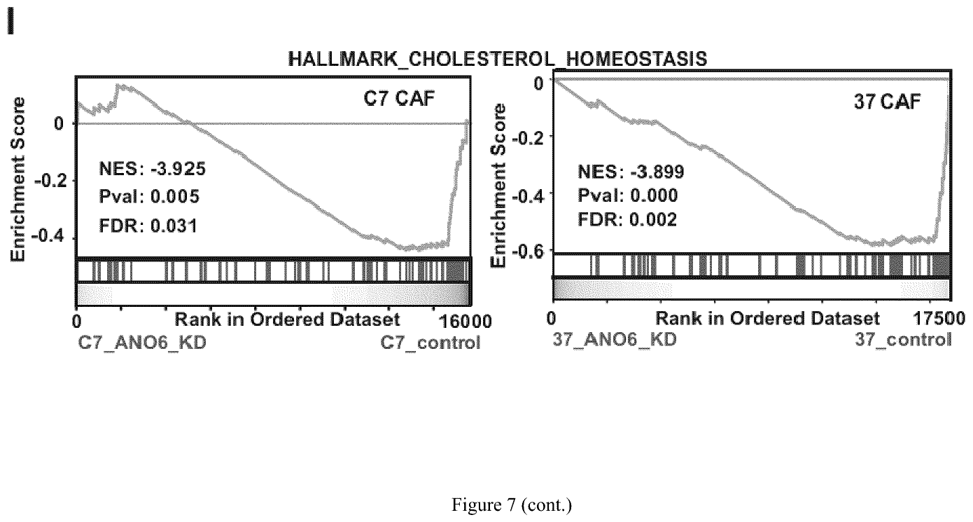

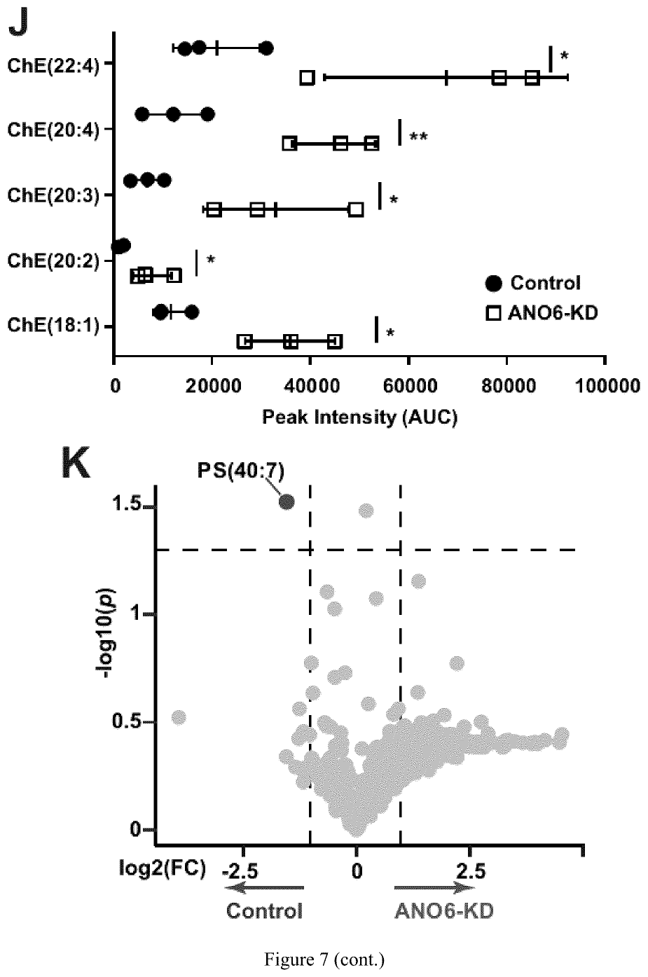

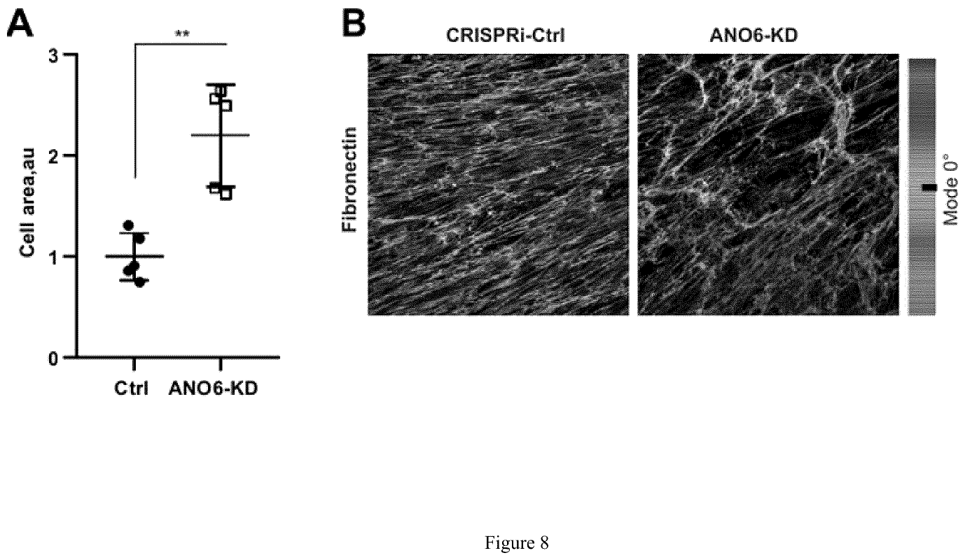

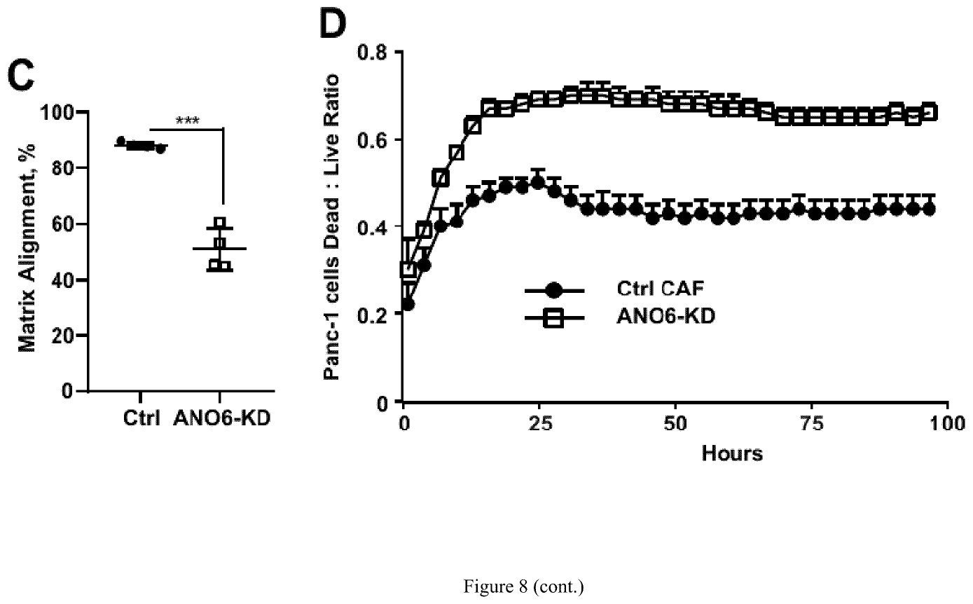

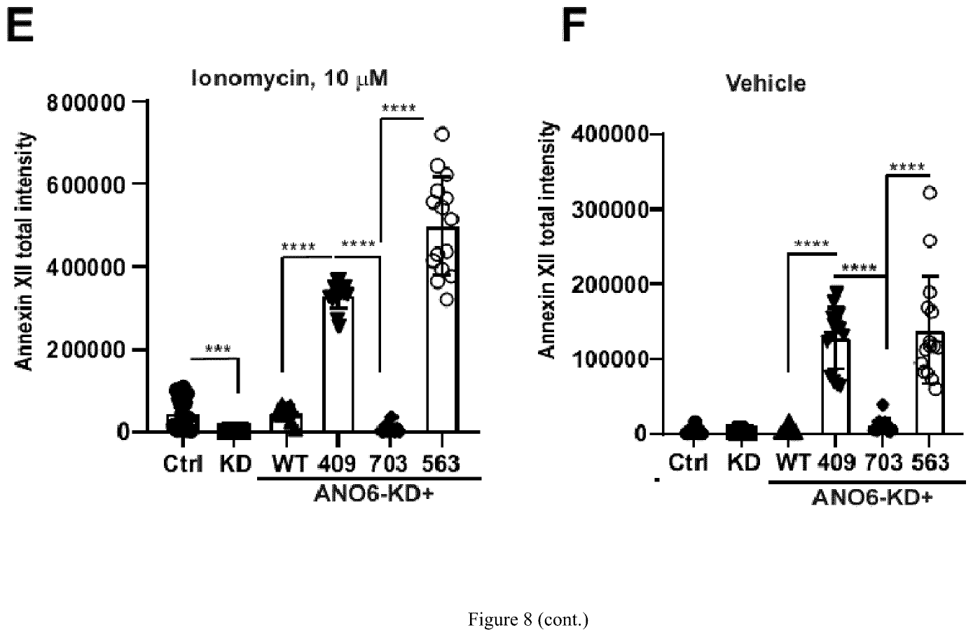

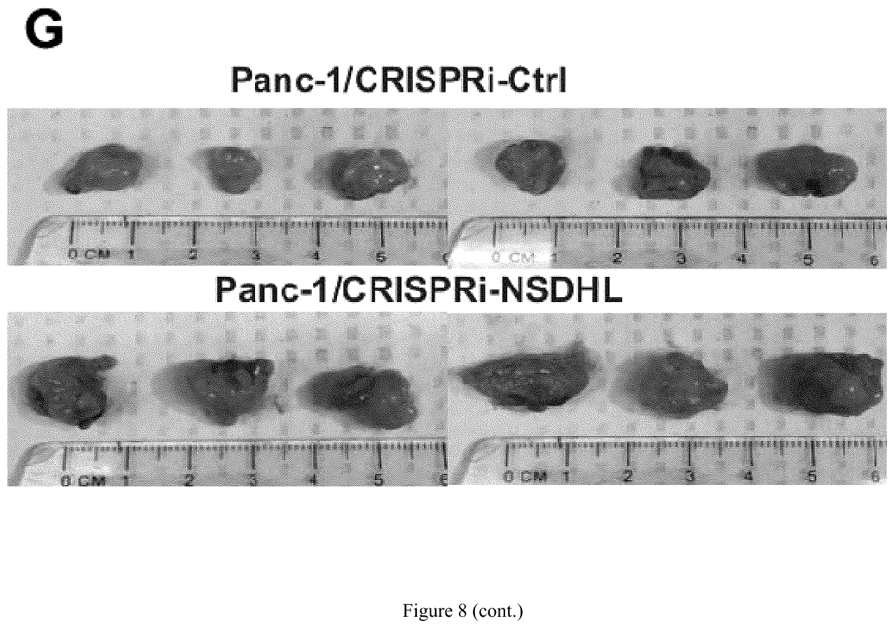

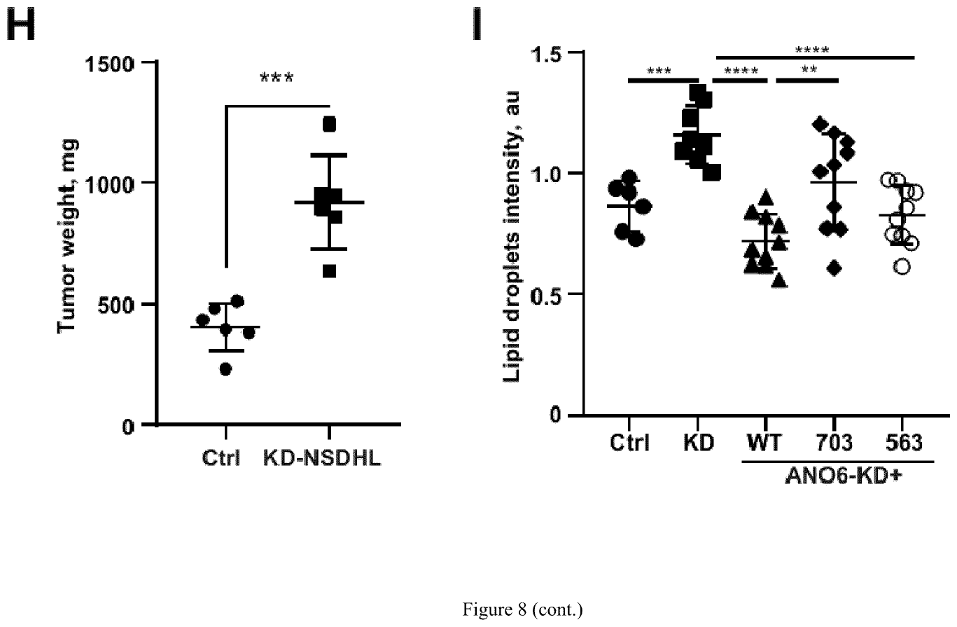

Cholesterol delivery to cancer cells is critical for tumor-promoting function of ANO6 in CAFs. It was next determined whether ANO6 in CAFs is critical for the ability to provide cholesterol and support the viability of cancer cells in co-cultures in lipid-poor media and in vivo. To better understand how ANO6 influences the intrinsic biology of CAFs, the features of CAFs was compared with CRISPRi-depleted ANO6-KD compared to non-targeting gRNA-depleted control cells. ANO6-KD fibroblasts exhibit a slower growth rate (FIG. 7, Panel A), acquire a stellate morphology (FIG. 7, Panel B, and FIG. 8, Panel A), and accumulate lipid droplets (FIG. 7, Panel B and Panel C), a feature of pancreatic stellate cells (PSCs) that have not been activated to CAFs by proximity to pancreatic cancer cells (Helms et al., Cancer Discov., 2022, 12, 484-501; and Shi et al., Nature, 2019, 569, 131-135). Furthermore, ANO6 depletion abrogated the ability of CAFs to produce structurally organized extracellular matrix characterized by fibronectin fibers aligned in parallel patterns (FIG. 8, Panel B and Panel C) (Franco-Barraza et al., Elife, 2017, 6). In contrast to PSCs, CAFs typically activate the integrin signaling pathway, which is reflected by autophosphorylation of focal adhesion kinase (FAK) and AKT (Franco-Barraza et al., Elife, 2017, 6). ANO6-KD fibroblastic cells exhibit markedly reduced levels of autophosphorylated focal adhesion kinase (pY397) FAK, and (pS473) AKT (FIG. 7, Panel D).

Importantly, ANO6 inactivation in CAFs reduces the viability of NSDHL-depleted MiaPaCa-2^{ΔNSDHL} (FIG. 7, Panel E) and Panc-1^{ΔNSDHL} (FIG. 7, Panel F, and FIG. 8, Panel D) PDAC cells co-cultured with CAFs in LDS-containing media. Furthermore, introduction of the wild-type murine mCherry-tagged ANO6 to ANO6-KD human CAFs restored PtdSer externalization in CAFs (FIG. 8, Panel E and Panel F), and rescued the viability of co-cultured Panc-1 cells, whereas inactivating D703R mutation in the ANO6 calcium-binding pocket (Arndt et al., Nat. Commun., 2022, 13, 6692; and Le et al., Nat. Commun., 2019, 10, 1846) showed no response to ionomycin (FIG. 8, Panel E) and resulted in lower viability of Panc-1 cells in LDS co-cultures compared to co-cultures with the wild type ANO6, or with the constitutively active ANO6-Y563A (FIG. 7, Panel F, and FIG. 8, Panel E and Panel F). Importantly, co-implantation of control CAFs with more aggressive cholesterol auxotroph Panc-1^{ΔNSDHL} cells (FIG. 8, Panel G and Panel H) resulted in doubling of orthotopic tumor weights, whereas ANO6-null CAFs failed to promote the growth of xenografts (FIG. 7, Panel G). These results support that CAF-expressed ANO6 plays a critical role in maintaining the growth of cancer cells through trogocytosis-mediated transfer of CAF membrane cholesterol.

It was next investigated whether inactivation of ANO6-dependent trogocytosis causes perturbations of cholesterol metabolism in CAFs as reflected in the accumulation of lipid droplets (FIG. 7, Panel B and Panel C) (Dumesnil et al., Nat. Commun., 2023, 14, 915). Indeed, the findings of reduced expression of nuclear SREBP1 fragment (FIG. 7, Panel D) were corroborated by reduced expression of Hallmark cholesterol metabolism signature genes in the transcriptomes of ANO6-KD fibroblasts compared to controls consistent with the acquired quiescence of ANO6-KD fibroblastic cells (FIG. 7, Panel H and Panel I). Furthermore, an increase in the abundance of cholesteryl esters in ANO6-KD fibroblasts (FIG. 7, Panel J) was discovered (Oni et al., J. Exp. Med., 2020, 217, 9), whereas introduction of the wild type or hyperactive variants of ANO6 reduced the abundance of lipid droplets, whereas the inactive D703R ANO6 mutant did not (FIG. 8, Panel I).

Comparison of lipids extracted from Panc-1 cells co-cultured with fibroblasts demonstrated abundance of PtdSer (40:7) as the only phospholipid predominant in Panc-1 cells co-cultured with control CAFs as compared to ANO6-KD (FIG. 7, Panel K). This finding supports the notion that activation of ANO6 in CAFs promotes trogocytic transfer of PM enriched in externalized PtdSer.

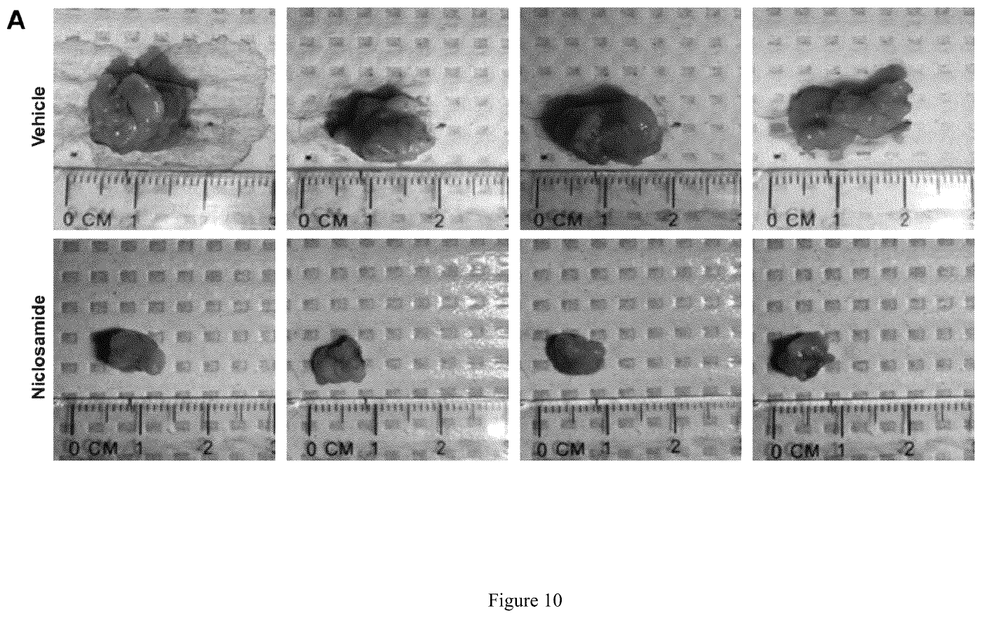

It was also determined if ANO6 blockade antagonizes cancer cell growth in orthotopic tumor grafts via blockade of lipids delivery. Treatment of orthotopic syngeneic pancreatic tumors with ANO6 inhibitors niclosamide (FIG. 9, Panel A and Panel B, and Figure FIG. 10, Panel A) or clofazimine (FIG. 9, Panel A and Panel C, and FIG. 10, Panel B) resulted in significant reduction of tumor weights. Treatment with niclosamide suppressed accumulation in the DsRed-tagged PDAC KPC3 cells (Gabitova-Comell et al., Cancer Cell, 2020, 38, 567-583) of intravenously administered LDL particles carrying BODIPY-cholesterol while the control vehicle-treated tumors showed 6-fold progressive enrichment of BODIPY-cholesterol fluorescence in cancer cells at 16 hours post intravenous injection (FIG. 9, Panel D). In analyses of intratumoral vesicles containing BODIPY-cholesterol, progressive vesicles accumulation in vehicle-treated orthotopic tumors at 19 hours post injection which was dramatically reduced in niclosamide treated mice was also determined (FIG. 9, Panel E and Panel F).

### Example 5: Membrane Ruffling

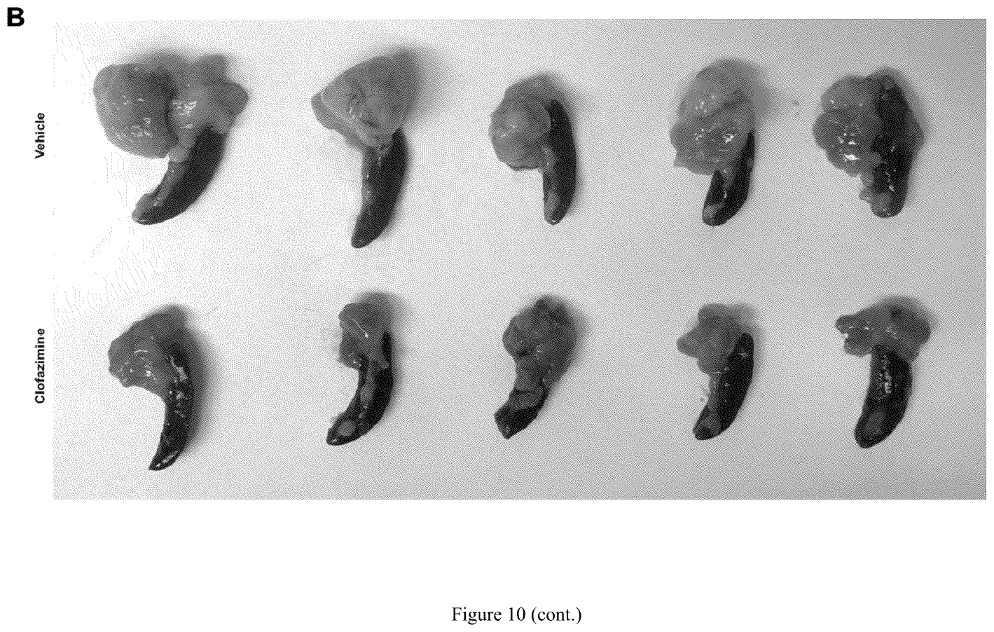

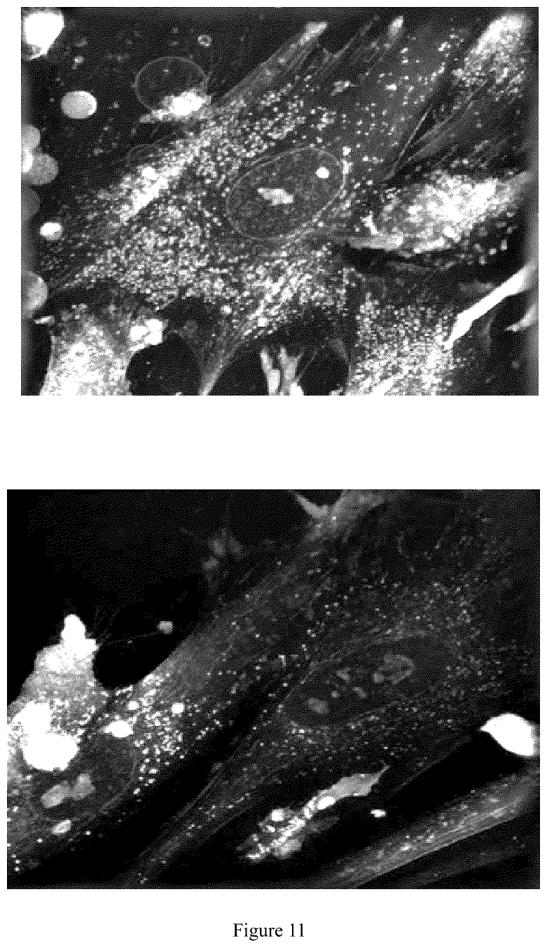

Referring to FIG. 11, CAFs KD TMEM16F (right) show no membrane ruffling compared to CAFs KD Control (left).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A pharmaceutical composition comprising:
a TMEM16F inhibitor;
an anti-cancer agent; and
a pharmaceutically acceptable carrier.

2. The pharmaceutical composition of claim 1, wherein the TMEM16F inhibitor is niclosamide, clofazimine, nitazoxanide, hexachlorophene, 10 bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

3. The pharmaceutical composition of claim 1, wherein the TMEM16F inhibitor is niclosamide, clofazimine, or nitazoxanide, or any combination thereof.

4. The pharmaceutical composition of claim 1, wherein the anti-cancer agent is methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, or docetaxel, or any combination thereof.

5. The pharmaceutical composition of claim 1, wherein the TMEM16F inhibitor is present in an amount from about 1 mg to about 1000 mg, from about 50 mg to about 800 mg, from about 75 mg to about 600 mg, from about 100 mg to about 500 mg, or form about 200 to about 400 mg.

6. The pharmaceutical composition of claim 1, wherein the pharmaceutical composition is an oral dosage formulation, an intravenous dosage formulation, a topical dosage formulation, an intraperitoneal dosage formulation, or an intrathecal dosage formulation.

7. A method of treating cancer in a mammal, the method comprising administering to the mammal a TMEM16F inhibitor.

8. The method of claim 7, wherein the TMEM16F inhibitor is niclosamide, clofazimine, nitazoxanide, hexachlorophene, 10 bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

9. The method of claim 7, wherein the TMEM16F inhibitor is niclosamide, clofazimine, or nitazoxanide, or any combination thereof.

10. The method of claim 7, wherein the amount of the TMEM16F inhibitor administered to the mammal is from about 1 mg to about 1000 mg.

11. The method of claim 7, wherein the mammal is also administered radiation therapy, an anti-cancer agent, or a combination thereof.

12. The method of claim 11, wherein the anti-cancer agent is methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, or docetaxel, or any combination thereof.

13. The method of claim 7, wherein the mammal is human.

14. The method of claim 7, wherein the cancer is lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma.

15. The method of claim 7, wherein the cancer is pancreatic cancer.
